(19) United States
(12) Patent Application Publication
(10) Pub. No.: US 2024/0261469 A1
(43) Pub. Date: Aug. 8, 2024

(54) METHODS FOR PROMOTING OSTEOCHONDRAL CELL AND TISSUE GROWTH INCLUDING IN LARGE LESIONS AND IN POPULATIONS AT RISK

(71) Applicant: CARTIHEAL (2009) LTD., Kfar Saba (IL)

(72) Inventor: Nir Altschuler, Hod Hasharon (IL)

(73) Assignee: CARTIHEAL (2009) LTD., Kfar Saba (IL)

(21) Appl. No.: 18/425,745

(22) Filed: Jan. 29, 2024

Related U.S. Application Data
(63) Continuation of application No. PCT/IL2022/050713, filed on Jul. 4, 2022.
(60) Provisional application No. 63/228,620, filed on Aug. 3, 2021.

(51) Int. Cl. A61L 27/36; A61L 27/56

(52) U.S. Cl. CPC A61L 27/3645; A61L 27/3604; A61L 27/56

(57) ABSTRACT

This invention provides optimized solid substrates for promoting cell or tissue growth or restored function and uses of same, which in some aspects address a need in populations at greater risk for osteochondral, bone or cartilage disease, or in some embodiments, in subjects for whom no effective therapeutic or surgical intervention exists, or in some embodiments, in subjects for whom recovery from and/or quality of life after therapeutic intervention is not significantly improved, or not sustained over time, or a combination thereof. In some embodiments, the optimized solid substrates for promoting cell or tissue growth or restored function and uses of same, address a need in populations with therapeutic results significantly greater than the surgical standard of care. In some embodiments, the optimized solid substrates for promoting cell or tissue growth or restored function and uses of same, address treating or repairing a large osteochondral, bone or cartilage lesion site in a subject in need thereof.

### CROSS REFERENCE TO RELATED APPLICATIONS

This application is a continuation of International Application Serial No. PCT/IL2022/050713, filed Jul. 4, 2022, which claims the benefit of and priority to U.S. Provisional Patent Application Ser. No. 63/228,620, filed Aug. 3, 2021, the disclosures of which are incorporated herein by reference.

### BACKGROUND OF THE INVENTION

Defects and degeneration of the articular cartilage surfaces of joints causes pain and stiffness. Damage to cartilage which protects joints can result from either physical injury as a result of trauma, sports or repetitive stresses (e.g., osteochondral fracture, secondary damage due to cruciate ligament injury) or from disease (e.g. osteoarthritis, rheumatoid arthritis, aseptic necrosis, osteochondritis dissecans).

Osteoarthritis (OA) results from general wear and tear of joints, most notably hip and knee joints. Osteoarthritis is common in the elderly but, in fact, by age 40 most individuals have some osteoarthitic changes in their weight bearing joints. Another emerging trend increasing the prevalence of osteoarthritis is the rise in obesity. The CDC estimates that 30% of American adults (or 60 million people) are obese. Obese adults are 4 times more likely to develop knee OA than normal weight adults Rheumatoid arthritis is an inflammatory condition which results in the destruction of cartilage. It is thought to be, at least in part, an autoimmune disease with sufferers having a genetic predisposition to the disease.

Orthopedic prevention and repair of damaged joints is a significant burden on the medical profession both in terms of expense and time spent treating patients. In part, this is because cartilage does not possess the capacity for self-repair. Attempts to re-grow hyaline cartilage for repair of cartilage defects remain unsuccessful. Orthopedic surgery is available in order to repair defects and prevent articular damage in an effort to forestall serious degenerative changes in a joint. The use of surgical techniques often requires the removal and donation of healthy tissue to replace the damaged or diseased tissue. Techniques utilizing donated tissue from autografts, allografts, or xenografts are wholly unsatisfactory as autografts add additional trauma to a subject and allografts and xenografts are limited by immunological reactivity to the host subject and possible transfer of infective agents. Surgical attempts to utilize materials other than human or animal tissue for cartilage regeneration have been unsuccessful.

Some surgical techniques to repair damaged joints include (i) arthroscopic debridement or lavage, (ii) marrow-stimulation techniques, such as microfracture, (iii) periosteal or perichondral grafting, (iv) autologous chondrocyte implantation, (v) osteochondral autograft transplantation, and (vi) osteochondral allograft transplantation. Each technique offers its own advantages and disadvantages, but with the setting of osteoarthritis with full-thickness chondral lesions, optimal treatment options are still lacking.

Ideal methods and/or materials, which restore tissue function and facilitates reconstruction of the morphology of such tissue across all populations in need remain elusive.

### SUMMARY OF THE INVENTION

This invention provides, in some aspects, for methods and uses for diagnostic procedures to identify subjects in at risk-populations, or subjects with severe disease/osteochondral defects whose treatment previously was accompanied by a poor or inferior prognosis, who can now be effectively identified and treated.

In other aspects, this invention provides for the surprising finding that solid substrates for use as described herein outperform the surgical standard of care therapeutic method of the subjects and populations described herein, including microfracture and debridement procedures, with a highly statistically significant improvement, over time.

In still other aspects, this invention provides for the surprising finding that subjects suffering from a BMI of over 30 kg/m2 do not have any successful treatment option for treating osteoarthritis in this subpopulation group can be identified/screened and now effectively treated with the methods/uses of this invention.

In still other embodiments, surprisingly, solid substrates do not require full packing to occupy all of, or as much of the space of the defect in the tissue as possible in order to be therapeutic and uniquely and unexpectedly, tissue remodeling was found in regions between implants, so that overall tissue re-modelling and regeneration occurred in these instances.

In some embodiments, the present invention provides a method of treating or repairing a large osteochondral, bone or cartilage lesion site in a subject in need thereof, said method comprising:
implanting two or more solid substrates comprising a coral or coral derivative within a region in said lesion site in said subject, wherein:
said defect site has a length or width or depth that is at least about 1.5 times greater than a length, width or depth of said two or more solid substrates being implanted therein;
said two or more solid substrates consisting essentially of two phases wherein:
   a first phase of said two phases comprises solid coral and said first phase further comprises a series of hollows along a longitudinal axis in said first phase;
   a second phase of said two phases comprises a solid coral;
said two or more solid substrates being characterized by a specific fluid uptake capacity value of at least 75% or characterized by having a contact angle value of less than 60 degrees, when in contact with a fluid; and
said two or more solid substrates are further characterized by each having at least one substantially flat cross section at a terminus of said solid substrate and tapered sides, which tapered sides are at an angle of from 0.75 to about 4 degrees from a longitudinal axis along said solid substrate.

In some embodiments, the present invention provides for two or more solid substrates comprising a coral or coral derivative for use in treating or repairing a large osteochondral, bone or cartilage lesion site in a subject in need thereof, wherein the defect site has a length or width or depth that is at least about 1.5 times greater than a length, width or depth of said two or more solid substrates being implanted therein; the two or more solid substrates consisting essentially of two phases wherein:
a first phase of said two phases comprises solid coral and said first phase further comprises a series of hollows along a longitudinal axis in said first phase;
a second phase of said two phases comprises a solid coral;
said two or more solid substrates being characterized by a specific fluid uptake capacity value of at least 75% or characterized by having a contact angle value of less than 60 degrees, when in contact with a fluid; and
said two or more solid substrates are further characterized by each having at least one substantially flat cross section at a terminus of said solid substrate and tapered sides, which tapered sides are at an angle of from 0.75 to about 4 degrees from a longitudinal axis along said solid substrate and wherein the two or more solid substrates are implanted within a region in the lesion site in the subject.

In some embodiments, according to these aspects, tissue remodeling may occur in tissue regions located between the implanted substrates, so that overall tissue re-modelling and regeneration is provided, as well, representing embodied aspects of this invention.

In some embodiments, the invention provides a method of treating osteochondral, bone or cartilage disease in a subject from a population at greater risk for same, said method comprising:
Identifying a subject from a population at greater risk for osteochondral, bone or cartilage disease and selecting said subject in need of treatment;
Implanting at least one solid substrate comprising a coral or coral derivative within at least one affected region in bone, cartilage or osteochondral tissue in said subject, wherein:
said at least one solid substrate being characterized by a specific fluid uptake capacity value of at least 75% or characterized by having a contact angle value of less than 60 degrees, when in contact with a fluid; and
said at least one solid substrate is further characterized by each having at least one substantially flat cross section at a terminus of said solid substrate and tapered sides, which tapered sides are at an angle of from 0.75 to about 4 degrees from a longitudinal axis along said solid substrate.

In some embodiments, the invention provides a method of identifying a subject from a population at greater risk for osteochondral, bone or cartilage disease for diagnosis and/or prognosis of the subject, to improve access to treatment for the subject, said method comprising:
Identifying a subject from a population at greater risk for osteochondral, bone or cartilage disease ordinarily not provided any treatment option and selecting said subject in need of treatment, wherein the treatment entails the use of
at least one solid substrate comprising a coral or coral derivative, wherein:
said at least one solid substrate being characterized by a specific fluid uptake capacity value of at least 75% or characterized by having a contact angle value of less than 60 degrees, when in contact with a fluid; and
at least one solid substrate is further characterized by each having at least one substantially flat cross section at a terminus of said solid substrate and tapered sides, which tapered sides are at an angle of from 0.75 to about 4 degrees from a longitudinal axis along said solid substrate; wherein
the solid substrate is suitable for use in the subject.

In some embodiments, the invention provides a method of personalized medical treatment of an osteochondral, bone or cartilage disease in a subject from a population at greater risk for same, said method comprising:
Identifying a subject from a population at greater risk for osteochondral, bone or cartilage disease and selecting said subject in need of treatment;
Implanting at least one solid substrate comprising a coral or coral derivative within at least one affected region in bone, cartilage or osteochondral tissue in said subject, wherein:
said at least one solid substrate being characterized by a specific fluid uptake capacity value of at least 75% or characterized by having a contact angle value of less than 60 degrees, when in contact with a fluid; and
said at least one solid substrate is further characterized by each having at least one substantially flat cross section at a terminus of said solid substrate and tapered sides, which tapered sides are at an angle of from 0.75 to about 4 degrees from a longitudinal axis along said solid substrate.

In some embodiments, the population at greater risk is an age bracket of 50 years or more and in some embodiments, the population at greater risk is an age bracket of 60 years or more.

In some embodiments, the d method promotes significant cartilage regeneration, healing or a combination thereof in said subject within six months as compared to microfracture and debridement procedures conducted in subjects in the same age bracket. In some embodiments, the population at greater risk is female and in some embodiments, the population at greater risk is post-menopausal females.

In some embodiments, the method promotes significant cartilage regeneration, healing or a combination thereof in said subject within six months as compared to microfracture and debridement procedures conducted in female subjects.

In some embodiments, the population at greater risk has a body mass index (BMI) of 30 kg/m2 or more and in some embodiments, the method promotes significant cartilage regeneration, healing or a combination thereof in said subject within six months of said treatment, or in some embodiments, within twelve months of said treatment, or in some embodiments, within eighteen months of said treatment. In some embodiments, according to this aspect, microfracture and debridement procedures are contraindicated in said population.

As exemplified and described further herein, uniquely the methods of this invention are directed to the surprising finding that solid substrates for use as described herein outperformed the surgical standard of care therapeutic method of the subjects and populations described herein, including microfracture and debridement procedures, with a highly statistically significant improved KOOS scores, over time.

Uniquely as exemplified and described herein, solid substrates for use as described herein were therapeutic in subjects/populations, for which there is no other available method of intervention or where intervention is contraindicated, for example, in subjects having a body mass index (BMI) of 30 kg/m2 or more.

Uniquely as exemplified and described herein, solid substrates for use as described herein were therapeutic in subjects/populations, in a sustained manner, with continued improvement over time, whereas it is known (and in some aspects demonstrated herein) that the surgical standard of care (SSOC) methods, for example, of microfracture and debridement, decline in their efficacy and/or sustainability over time, for example, after 6 or after 12 or after 18 months, or after 24 months.

Uniquely as exemplified and described herein, solid substrates for use as described herein were therapeutic in subjects/populations with an otherwise typically high failure rate for any of the existing treatment methods for cartilage and/or bone disease or a combination thereof.

Uniquely as exemplified and described herein, solid substrates for use as described herein were therapeutic in subjects/populations with large defects, for example, of a size of 3 cm² or more, wherein previously such large defects were more typically addressed by surgical prosthesis/replacement methods.

Uniquely as exemplified and described herein, solid substrates did not require full packing to occupy all of, or as much of the space of the defect in the tissue as possible in order to be therapeutic and uniquely and unexpectedly, tissue remodeling was found in regions between implants, so that overall tissue re-modelling and regeneration occurred in these instances.

All publications, patents, and patent applications mentioned herein are hereby incorporated by reference in their entirety as if each individual publication or patent was specifically and individually indicated to be incorporated by reference. In case of a conflict between the specification and an incorporated reference, the specification shall control. Where number ranges are given in this document, endpoints are included within the range. Furthermore, it is to be understood that unless otherwise indicated or otherwise evident from the context and understanding of one of ordinary skill in the art, values that are expressed as ranges can assume any specific value or sub-range within the stated ranges, optionally including or excluding either or both endpoints, in different embodiments of the invention, to the tenth of the unit of the lower limit of the range, unless the context clearly dictates otherwise. Where a percentage is recited in reference to a value that intrinsically has units that are whole numbers, any resulting fraction may be rounded to the nearest whole number.

### BRIEF DESCRIPTION OF THE DRAWINGS

FIG. 1 depicts the cumulative treatment failure rate as described herein.

FIGS. 2 and 3 graphically summarize the observed means and mean changes, respectively, including standard errors, as described herein.

FIG. 4 and FIG. 5 graphically summarize mean values over time and mean changes from baseline in KOOS Overall Score over time in the FAS for subjects with a K-L Grade of 2 or 3.

FIG. 6 and FIG. 7 graphically summarize mean values over time and mean changes from baseline in KOOS Overall Score over time in the FAS for subjects with total lesion area>3 cm2.

FIGS. 8-14 provide graphical summaries of the values and changes from baseline over time for confirmatory secondary endpoints of KOOS Pain, KOOS Quality of Life, and KOOS ADL scores, as well as the percentages of subjects achieving at least a 30-point improvement in KOOS Overall Score.

FIGS. 15, 16, 17 and 18 depict graphical analyses of KOOS Other Symptoms Score and KOOS Sports Score values and changes over time.

FIGS. 19A-19E depict representative X-ray and MRI results in individual subjects treated by embodied, illustrative, non-limiting methods of this invention.

FIGS. 20A-20F depict representative X-ray and MRI results in individual subjects treated by embodied, illustrative, non-limiting methods of this invention.

FIGS. 21A-21H depict representative X-ray and MRI results in individual subjects treated by embodied, illustrative, non-limiting methods of this invention.

### DETAILED DESCRIPTION OF THE PRESENT INVENTION

This invention provides, inter alia, optimized solid substrates for promoting cell or tissue growth or restored function and uses of same, which in some aspects address a need in populations at greater risk for osteochondral, bone or cartilage disease, or in some embodiments, in subjects for whom no effective therapeutic or surgical intervention exists, or in some embodiments, in subjects for whom recovery from and/or quality of life after therapeutic intervention is not significantly improved, or not sustained over time, or a combination thereof.

In some embodiments, the optimized solid substrates for promoting cell or tissue growth or restored function and uses of same, address a need in populations with therapeutic results significantly greater than the surgical standard of care.

In some embodiments, the optimized solid substrates for promoting cell or tissue growth or restored function and uses of same, address treating or repairing a large osteochondral, bone or cartilage lesion site in a subject in need thereof.

As is described further hereinunder, the mean improvement was clinically and statistically significantly larger for Agili-C™ as compared to the surgical standard of care (SSOC) methodology used as a comparator, even considering the results obtained by Month 6.

Furthermore, unexpectedly, not only was the mean improvement was clinically and statistically significantly larger for Agili-C™ earlier, but the effect was sustained over time, in marked contrast to that of SSOC, this even despite the fact that the Agili-C™ treated group had a larger number of subjects in this group with more severe disease/defect at the outset of the trial.

Still further unexpected and remarkable was the finding that a significantly higher rate of treatment failures was observed in the SSOC group as compared to the Agili-C™ treated subjects, and when the fact that the Agili-C™ treated group had a larger number of subjects in this group with more severe disease/defect at the outset of the trial, this finding is quite unexpected.

It is also noteworthy that 17.9% SSOC subjects required intra-articular injection to address persistent pain following the procedure, whereas only 1.8% Agili-C™ subjects required same, indicating that SSOC procedures that were not considered “failures” nonetheless far less adequately controlled the patient's pain, in marked contrast to patients provided Agili-C™.

Agili-C™'s superiority in effectiveness relative to standard of care confirmed across all subgroups defined by pre-specified covariates. Factors, such as subjects' activity level, BMI, status of ACL and meniscus, age, type of lesion, size of lesion or number of lesions—which could be expected to negatively impact treatment outcomes due to challenging conditions, did not negatively impact the Agili-C™ superiority over the current surgical standard of care, microfracture and debridement.

A number of unexpected findings arise from the instant invention(s).

In some aspects of the invention, there is a significant increased percentage of articular defect fill as evidenced by MRI at 12 and 24 months and a significant improvement in the overall KOOS score (as measuring Pain, symptoms, quality of life (QOL) ADL and ability to participate in Sport activity at 6, 12 and 18 months and methods/uses/systems/kits promoting same using the solid substrates of this invention are therefore contemplated applications of this invention.

In some embodiments, there is a significant increased improvement over baseline in IKDC subjective knee evaluation at 12, 18 and 24 months and methods/uses/systems/kits promoting same using the solid substrates of this invention are therefore contemplated applications of this invention.

In some embodiments, there is a significant increased improvement over baseline in Tegner score at 12, 18 and 24 months and methods/uses/systems/kits promoting same using the solid substrates of this invention are therefore contemplated applications of this invention.

In some embodiments, there is a significant increased improvement over baseline in QOL as determined by SF-12 v2 at 6, 12, 18 and 24 months post implantation and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with chondral lesions, and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with osteochondral lesions and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with a single lesions and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with multiple lesions and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with osteoarthritis and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients without osteoarthritis and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with lesions of up to and including 3 cm² in size and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with lesions of over 3 cm² in size and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients without previous ligament reconstruction and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients with an intact meniscus and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients having undergone a previous partial meniscectomy and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients undergoing a concomitant partial meniscectomy and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients leading an active lifestyle and methods/uses/systems/kits promoting same using the solid substrates of this invention are therefore contemplated applications of this invention.

In some embodiments, there is a significant increased improvement from baseline to 24 months in the average KOOS score (pain, symptoms, QOL, ADS and participation in sports activities) in patients leading an inactive lifestyle and methods/uses/systems/kits promoting same using the solid substrates of this invention are therefore contemplated applications of this invention.

Accordingly, this invention provides methods of treating or repairing an osteochondral, bone or cartilage lesion site in a subject in need thereof, which method comprises improvement in accordance with any of the following parameters:
a significant increased percentage of articular defect fill as evidenced by MRI at from about 12 through about 24 months;
a significant improvement in the overall KOOS score (as measuring Pain, symptoms, quality of life (QOL) ADL and ability to participate in Sport activity) at from about 6, 12 and 18 months;
a significant increased improvement over baseline in IKDC subjective knee evaluation at from about 12, 18 and 24 months;
a significant increased improvement over baseline in Tegner score at from about 12, 18 and 24 months;
a significant increased improvement over baseline in QOL as determined by SF-12 v2 at from about 6, 12, 18 and 24 months;
a significant increased improvement from baseline to about 24 months in the average KOOS score (pain, symptoms, QOL, ADS and participation in sports activities) in patients with chondral lesions;
a significant increased improvement from baseline to about 24 months in the average KOOS score (pain, symptoms, QOL, ADS and participation in sports activities) in patients with osteochondral lesions;
a significant increased improvement from baseline to about 24 months in the average KOOS score (pain, symptoms, QOL, ADS and participation in sports activities) in patients with a single lesions;
a significant increased improvement from baseline to about 24 months in the average KOOS score (pain, symptoms, QOL, ADS and participation in sports activities) in patients with multiple lesions;
a significant increased improvement from baseline to about 24 months in the average KOOS score (pain, symptoms, QOL, ADS and participation in sports activities) in patients with osteoarthritis;
a significant increased improvement from baseline to about 24 months in the average KOOS score (pain, symptoms, QOL, ADS and participation in sports activities) in patients without osteoarthritis;
a significant increased improvement from baseline to about 24 months in the average KOOS score (pain, symptoms, QOL, ADS and participation in sports activities) in patients with lesions of up to and including 3 cm² in size;
a significant increased improvement from baseline to about 24 months in the average KOOS score (pain, symptoms, QOL, ADS and participation in sports activities) in patients with lesions of over 3 cm² in size;
a significant increased improvement from baseline to about 24 months in the average KOOS score (pain, symptoms, QOL, ADS and participation in sports activities) in patients without previous ligament reconstruction;
a significant increased improvement from baseline to about 24 months in the average KOOS score (pain, symptoms, QOL, ADS and participation in sports activities) in patients with an intact meniscus;
a significant increased improvement from baseline to 24 months in the average KOOS score (pain, symptoms, QOL, ADS and participation in sports activities) in patients having undergone a previous partial meniscectomy;
a significant increased improvement from baseline to about 24 months in the average KOOS score (pain, symptoms, QOL, ADS and participation in sports activities) in patients undergoing a concomitant partial meniscectomy;
a significant increased improvement from baseline to about 24 months in the average KOOS score (pain, symptoms, QOL, ADS and participation in sports activities) in patients leading an active lifestyle; and
a significant increased improvement from baseline to about 24 months in the average KOOS score (pain, symptoms, QOL, ADS and participation in sports activities) in patients leading an inactive lifestyle;

It will be understood that any method comprising implanting a solid substrate as herein defined in an affected tissue of a subject, whereby the specific parameters as described herein are improved as described, is to be considered a contemplated aspect of the invention.

In some embodiments, this invention provides a method of treating or repairing a large osteochondral, bone or cartilage lesion site in a subject in need thereof, said method comprising:
implanting two or more solid substrates comprising a coral or coral derivative within a region in said lesion site in said subject, wherein:
said defect site has a length or width or depth that is at least about 1.5 times greater than a length, width or depth of said two or more solid substrates being implanted therein;
said two or more solid substrates consisting essentially of two phases wherein:
   a first phase of said two phases comprises solid coral and said first phase further comprises a series of hollows along a longitudinal axis in said first phase;
   a second phase of said two phases comprises a solid coral;
said two or more solid substrates being characterized by a specific fluid uptake capacity value of at least 75% or characterized by having a contact angle value of less than 60 degrees, when in contact with a fluid; and
said two or more solid substrates are further characterized by each having at least one substantially flat cross section at a terminus of said solid substrate and tapered sides, which tapered sides are at an angle of from 0.75 to about 4 degrees from a longitudinal axis along said solid substrate.

According to this aspect and in some embodiments, the lesion site is larger than 3 cm² and in some embodiments, the two or more solid substrates are placed near opposite boundaries of said lesion area larger than 3 cm². In some embodiments, according to this aspect, the two or more solid substrates are placed at least at about a 3 mm distance from each other along a Cartesian axis and in some embodiments the two or more solid substrates are placed at spaced intervals of at least about 3 mm in distance to span said lesion area. In some embodiments, the two or more solid substrates do not fill more than 90% of the total area of said lesion site.

According to this aspect and in some embodiments, the method promotes cartilage regeneration, healing or a combination thereof in regions not in immediate contact with said region in which said solid substrates are placed within said lesion, thereby being a method of treating or repairing a large osteochondral, bone or cartilage lesion site in a subject in need thereof.

According to this aspect and in some embodiments, the microfracture and debridement procedures are contraindicated in said population.

According to this aspect and in some embodiments, the method promotes cartilage regeneration, healing or a combination thereof in regions not proximal to said region in which said solid substrates are placed within said lesion, thereby being a method of treating or repairing a large osteochondral, bone or cartilage lesion site in a subject in need thereof.

According to this aspect and in some embodiments, the method promotes significant cartilage regeneration, healing or a combination thereof in said subject within six months as compared to microfracture and debridement procedures.

According to this aspect and in some embodiments, the method promotes and/or sustains significant cartilage regeneration, healing or a combination thereof in said subject twelve months or more, as compared to microfracture and debridement procedures.

According to this aspect and in some embodiments, the method promotes bone regeneration, healing or a combination thereof in regions not proximal to said region in which said solid substrates are placed within said lesion.

According to this aspect and in some embodiments, the two or more solid substrates are positioned anywhere by or near the periphery of said lesion site and said two or more solid substrates do not substantially fill said lesion site.

According to this aspect and in some embodiments, the two or more solid substrates are positioned at or near a diseased or affected tissue site, whereby the solid substrates being implanted are also proximally located to healthy cartilage and/or bone tissue. According to this aspect, and in some embodiment, proximity of the solid substrates to healthy cartilage and/or bone tissue promotes the healing/treatment of the lesion, in some embodiments, by promoting migration of healthy cells and/or participation of therapeutic factors within the healthy tissue.

According to this aspect and in some embodiments, the two or more solid substrates are positioned at any relative position with respect to each other within said lesion site.

According to this aspect and in some embodiments, the subject suffers from a full or partial thickness articular cartilage defect; osteochondral defect; osteoarthritis; osteochondritis dissecans; a joint defect; or a defect resulting from trauma, sports, or repetitive stress.

In other embodiments, this invention provides a method of treating osteochondral, bone or cartilage disease in a subject from a population at greater risk for same, said method comprising:
Identifying a subject from a population at greater risk for osteochondral, bone or cartilage disease and selecting said subject in need of treatment;
Implanting at least one solid substrate comprising a coral or coral derivative within at least one affected region in bone, cartilage or osteochondral tissue in said subject, wherein:
said at least one solid substrate being characterized by a specific fluid uptake capacity value of at least 75% or characterized by having a contact angle value of less than 60 degrees, when in contact with a fluid; and
said at least one solid substrate is further characterized by each having at least one substantially flat cross section at a terminus of said solid substrate and tapered sides, which tapered sides are at an angle of from 0.75 to about 4 degrees from a longitudinal axis along said solid substrate.

According to this aspect and in some embodiments, the population at greater risk is an age bracket of 50 years or more, or in some embodiments, the population at greater risk is an age bracket of 60 years or more.

According to this aspect and in some embodiments, the method promotes significant cartilage regeneration, healing or a combination thereof in said subject within six months as compared to microfracture and debridement procedures conducted in subjects in the same age bracket.

According to this aspect and in some embodiments, the population at greater risk is female and in some embodiments, the population at greater risk is post-menopausal females.

According to this aspect and in some embodiments, the method promotes significant cartilage regeneration, healing or a combination thereof in said subject within six months as compared to microfracture and debridement procedures conducted in female subjects.

According to this aspect and in some embodiments, the population at greater risk has a body mass index (BMI) of 30 kg/m2 or more. According to this aspect and in some embodiments, the method promotes significant cartilage regeneration, healing or a combination thereof in said subject within six months of said treatment, or in some embodiments, the method promotes significant cartilage regeneration, healing or a combination thereof in said subject within twelve months of said treatment, or in some embodiments, the method promotes significant cartilage regeneration, healing or a combination thereof in said subject within eighteen months of said treatment. According to this aspect and in some embodiments, the microfracture and debridement procedures are contraindicated in said population.

According to this aspect and in some embodiments, the method comprises implanting two or more of said solid substrates and in some embodiment, the two or more solid substrates are placed at least at about a 3 mm distance along a cartesian axis from each other. According to this aspect and in some embodiments, the two or more solid substrates are placed at spaced intervals of at least about 5 mm in distance to span said lesion area larger than 3 cm2. According to this aspect and in some embodiments, the method promotes cartilage regeneration, healing or a combination thereof even in regions not proximal to said region in which said solid substrates are implanted.

According to this aspect and in some embodiments, the two or more solid substrates are positioned at any relative position with respect to each other within said lesion site.

In some embodiments, the invention provides an optimized solid substrate for use in accordance with the methods and as part of a kit for uses as described herein, which solid substrate comprises a coral or coral derivative, is characterized by a specific fluid uptake capacity value of at least 75% or is characterized by having a contact angle value of less than 60 degrees, when in contact with a fluid and which is further characterized by tapered sides.

In some embodiments, the invention provides an optimized solid substrate for use in accordance with the methods and as part of a kit for uses as described herein, which solid substrate comprises a porous coral substrate, characterized by being absorptive of biologic fluids when implanted in situ, is of sufficient strength and hardness and useful in stimulating bone and/or cartilage repair and which substrate is further characterized by tapered sides.

In some aspects, the porous natural substrate may be acellular or further processed to be suitable for implantation within a human host.

In some aspects, a solid substrate as herein described may be characterized by comprising tapered sides. In some embodiments, the term “tapered sides” refers to the sides of the solid implant being at an angle of from 0.75 to about 4 degrees from a longitudinal axis along said solid substrate.

In some aspects, the solid substrate will be characterized by at least one substantially flat cross section at a terminus of said solid substrate, as well.

In some aspects, the reference to being characterized by a substantially flat cross section of said solid substrate does not preclude the potential for rounded edges of the solid substrate, or in some embodiments, a slightly rounded top or bottom surface. In some embodiments, according to this aspect, the solid substrate may have slight bumps or other imperfections at either terminus. In some embodiments, according to this aspect, the solid substrate will be slightly rounded, but without a terminal point or pointed end or ends. In some embodiments, one terminus may be more rounded in appearance than another. In some embodiments, a terminus may be further characterized by the presence of a series of longitudinal channels or voids created therein, however, the top surface may still be considered to be substantially flat, as the surface in overall appearance will be substantially flat.

In some aspects, the solid substrate will have a substantially conical shape.

In some aspects, the term “a substantially conical” with regard to shape refers to a solid substrate characterized as above, with a shape approximating a cone in that it possesses a circular cross section at each terminus of the substrate, and tapered sides. In some aspects, the term “a substantially conical” precludes the presence of a terminal sharp point in the substrate, but does encompass a shape approximating a cone shape, whereby a pointy end is shaved or removed, leaving a circular cross section, tapered end in its stead.

According to this aspect, and in some embodiments, the solid substrate is characterized by a conical frustum shape.

According to this aspect, and in some embodiments, the solid substrate is characterized by a conical frustum shape, i.e. a portion of a solid cone that lies between two parallel planes cutting same. In some aspects, the diameter of the two parallel planes cutting the solid cone differs, such that one is larger and one is smaller. In some embodiments, the solid substrate characterized by a conical frustum shape will be further characterized by insertion of the solid substrate within an osteochondral defect such that the plane characterized with a smaller diameter is inserted first, such that the plane characterized by the larger diameter is most apically located within the implantation site.

In some aspects, the solid substrate will have a substantially pyramidal shape.

In some aspects, the term “a substantially pyramidal” with regard to shape refers to a solid substrate characterized as above, with a shape approximating a pyramid in that it possesses a flat cross section at each terminus of the substrate, and tapered sides. In some aspects, the term “substantially pyramidal” precludes the presence of a terminal sharp point in the substrate, but does encompass a shape approximating a pyramid shape, whereby a pointy end is shaved or removed, leaving a flat cross section, tapered end in its stead.

According to this aspect, and in some embodiments, the solid substrate is characterized by a pyramidal frustum shape.

According to this aspect, and in some embodiments, the solid substrate is characterized by a pyramidal frustum shape, i.e. a portion of a solid pyramid that lies between two parallel planes cutting same. In some aspects, the length/width of the two parallel planes cutting the solid pyramid differs, such that one is larger and one is smaller. In some embodiments, the solid substrate characterized by a pyramidal frustum shape will be further characterized by insertion of the solid substrate within an osteochondral defect such that the plane characterized with a smaller length/width is inserted first, such that the plane characterized by the larger length/width is most apically located within the implantation site.

In some embodiments, the solid substrate is characterized by a substantially ovoid shape, when referring to a shape regarding the boundaries or outer contour of the substrate.

In some aspects, the solid substrate is characterized by any shape, that permits tapered sides, and in some embodiments, substantially flat termini, which can accommodate an ideal, optimized press fit within a defect site. In some aspects, the solid substrate will assume any appropriate geometry approximating a bar, cube, oval, with tapered sides, i.e. a solid shape substantially resembling for example, a bar, cube or oval, with two parallel planes cutting same.

In some aspects, the solid substrate is characterized by a shape with tapered sides as described, that can approximate the overall shape of a talus, great toe, shoulder, condyle, ankle, patella, trochlea, pelvis, vertebra, hip and others, as will be appreciated by the skilled artisan, or approximate a smaller piece of same that can insert within such structures readily, and in an optimized press fit manner.

In some aspects, the solid substrate may be characterized by having a first end with a diameter varying in size of between about 50-95% from that of a second diameter of the second end of the substrate, or in some embodiments, the solid substrate may be characterized by having a first end with a diameter varying in size of between about 50-65% from that of a second diameter of the second end of the substrate, or having a first end with a diameter varying in size of between about 55-75% from that of a second diameter of the second end of the substrate, having a first end with a diameter varying in size of between about 70-85% from that of a second diameter of the second end of the substrate, having a first end with a diameter varying in size of between about 75-95% from that of a second diameter of the second end of the substrate, having a first end with a diameter varying in size of between about 60-95% from that of a second diameter of the second end of the substrate, having a first end with a diameter varying in size of between about 65-95% from that of a second diameter of the second end of the substrate, having a first end with a diameter varying in size of between about 80-98% from that of a second diameter of the second end of the substrate having a first end with a diameter varying in size of between about 70-85% from that of a second diameter of the second end of the substrate.

In some aspects, the tapered sides are at an angle of two degrees from a longitudinal axis along the solid substrate.

In some aspects, the tapered sides are at an angle of 0.5 to 4.5 degrees from a longitudinal axis along the solid substrate. In some aspects, the tapered sides are at an angle of 0.5 to 4 degrees from a longitudinal axis along the solid substrate. In some aspects, the tapered sides are at an angle of 0.75 to 3.5 degrees from a longitudinal axis along the solid substrate, or in some embodiments, the tapered sides are at an angle of 1 to 3.25 degrees from a longitudinal axis along the solid substrate, or in some embodiments, the tapered sides are at an angle of 1.5 to 2.75 degrees from a longitudinal axis along the solid substrate, or in some embodiments, the tapered sides are at an angle of 1.75 to 4 degrees from a longitudinal axis along the solid substrate.

The solid substrates of this invention are characterized by a specific fluid uptake capacity value of at least 75%, which specific fluid uptake capacity value is determined by establishing a spontaneous fluid uptake value divided by a total fluid uptake value, or are characterized by having a contact angle value of less than 60 degrees, when in contact with a fluid. Methods for the determination of the specific fluid uptake and contact angle value have been described, for example, in PCT International Application Publication Number WO2014125478, hereby incorporated by reference in its entirety.

In some aspects, the solid substrate will be characterized by a curved cross section at a terminus of the solid substrate. According to this aspect, and in some embodiments, such curvature will be more typically at an apical surface of a solid substrate as herein described, in order to accommodate an appropriate fit of the implant, such that the correction of a defect containing a curved surface is readily accomplished. In some aspects, the curved surface of the defect site is substantially symmetrical and therefore the apical surface of the implant will comprise a substantially symmetrically curved surface. In some aspects, the curved surface of the defect site is substantially asymmetrical and therefore the apical surface of the implant will comprise a substantially asymmetrically curved surface.

In some embodiments, reference to a curved surface or curved cross section at a terminus of a solid substrate of this invention will include a radius of curvature of such substrate, where the radius may vary along an X-axis of a plane of a surface of such substrate, or in some embodiments, the radius may vary along a Z-axis of a plane of a surface of such substrate, or in some embodiments, radius may vary along an X-axis and a Z-axis of a plane of a surface of such substrate.

Similarly, and as described herein, reference to a curved surface or curved cross section at a terminus of a solid substrate of this invention will include a radius of curvature of such substrate, where the radius may vary along a coronal or sagittal plane of a surface of such substrate, or in some embodiments, such radius may vary along a lateral or anterior/posterior plane of a surface of such substrate, or in some embodiments, such radius may very along any axis as herein defined, along a surface of a substrate as herein described.

The solid substrates of this invention will, in some embodiments, comprise a coralline-based material. Coral, which is comprised of CaCO₃ in the crystalline form of aragonite or calcite has been shown to possess the advantage of supporting fast cellular invasion, adherence and proliferation. Coral has been shown to be an effective substrate for facilitation of the adherence, proliferation and differentiation of mesenchymal stem cells, and ultimate incorporation into cartilage and/or bone tissue. Coral has also been shown to serve as an excellent substrate for promoting adherence and proliferation of a number of other cell types, serving as an excellent support for cell and tissue growth.

The terms “coral” and “aragonite” and “calcite” may be used interchangeably herein.

In some embodiments, reference to an “implant” or “plug” or “solid substrate”, as used herein refers to any embodiment or combined embodiments as herein described with regard to the solid substrates and to be considered as being included in the described aspect of this invention. For example, reference to a “solid substrate” as used herein, is to be understood to refer to any embodiment of a solid substrate as described herein being applicable for the indicated purpose or containing the indicated attribute, etc.

In one embodiment, “solid substrate” refers to a shaped platform used for cell and/or tissue repair and/or restored function, wherein the shaped platform provides a site for such repair and/or restored function. In one embodiment, the solid substrate is a temporary platform. In one embodiment, “temporary platform” refers to a natural degradation of a coral of this invention that occurs over time during such repair, wherein the natural fully or partially degradation of the coral may results in a change of solid substrate shape over time and/or change in solid substrate size over time.

In some embodiments, the solid implant is cannulated and in some embodiments, the solid implant is not cannulated.

In one embodiment, the coral is from the implant comprises a coral of a Poritesspecies. In one embodiment, the coral is Porites Lutea. In one embodiment, the coral is from the Acropora species. In one embodiment, the coral is Acropora grandis, which in one embodiment is very common, fast growing, and easy to grow in culture. Thus, in one embodiment Acropora samples can be easily collected in sheltered areas of the coral reefs and collection from the coral reefs can be avoided by use of cultured coral material.

In another embodiment, the coral is from the Millepora species. In one embodiment, the coral is Millepora dichotoma. In one embodiment, the coral has a pore size of 150 μm and can be cloned and cultured, making Millerpora useful as a framework in the solid substrates, methods and/or kits of this invention.

In one embodiment, the coral is from the Goniopora species. In some embodiments, the coral is Goniopora albiconus, Goniopora burgosi, Goniopora cellulosa, Goniopora ceylon, Goniopora ciliatus, Goniopora columna, Goniopora djiboutiensis, Goniopora eclipsensis, Goniopora fruticosa, Goniopora gracilis, Goniopora klunzingeri, Goniopora lobata, Goniopora mauritiensis, Goniopora minor, Goniopora norfolkensis, Goniopora palmensis, Goniopora pandoraensis, Goniopora parvistella, Goniopora pearsoni, Goniopora pendulus, Goniopora planulata, Goniopora polyformis, Goniopora reptans, Goniopora savignyi, Goniopora somaliensis, Goniopora stokes, Goniopora stutchburyi, Goniopora sultani, Goniopora tenella, Goniopora tenuidens or Goniopora viridis.

In another embodiment, the coral is from any one or more of the following species Favites halicora; Goniastrea retiformis; Acanthastrea echinata; Acanthastrea hemprichi; Acanthastrea ishigakiensis; Acropora aspera; Acropora austera; Acropora sp. “brown digitate”; Acropora carduus; Acropora cerealis; Acropora chesterfieldensis; Acropora clathrata; Acropora cophodactyla; Acropora sp. “danai-like”; Acropora divaricata; Acropora donei; Acropora echinata; Acropora efflorescens; Acropora gemmifera; Acropora globiceps; Acropora granulosa; Acropora cf hemprichi; Acropora kosurini; Acropora cf loisettae; Acropora longicyathus; Acropora loripes; Acropora cf lutkeni; Acropora paniculata; Acropora proximalis; Acropora rudis; Acropora selago; Acropora solitaryensis; Acropora cf spicifera as per Veron; Acropora cf spicifera as per Wallace; Acropora tenuis; Acropora valenciennesi; Acropora vaughani; Acropora vermiculata; Astreopora gracilis; Astreopora myriophthalma; Astreopora randalli; Astreopora suggesta; Australomussa rowleyensis; Coscinaraea collumna; Coscinaraea crassa; Cynarina lacrymalis; Distichopora violacea; Echinophyllia echinata; Echinophyllia cf echinoporoides; Echinopora gemmacea; Echinopora hirsutissima; Euphyllia ancora; Euphyllia divisa; Euphyllia yaeyamensis; Favia rotundata; Favia truncatus; Favites acuticollis; Favities pentagona; Fungia granulosa; Fungia klunzingeri; Fungia mollucensis; Galaxea acrhelia; Goniastrea edwardsi; Goniastea mimuita; Hydnophora pilosa; Leptoseris explanata; Leptoseris incrustans; Leptoseris mycetoseroides; Leptoseris scabra; Leptoseris yabei; Lithophyllon undulatum; Lobophyllia hemprichii; Merulina scabricula; Millepora dichotoma; Millepora exaesa; Millipora intricata; Millepora murrayensis; Millipora platyphylla; Monastrea curta; Monastrea colemani; Montipora caliculata; Montipora capitata; Montipora foveolata; Montipora meandrina; Montipora tuberculosa; Montipora cf vietnamensis; Oulophyllia laevis; Oxypora crassispinosa; Oxypora lacera; Pavona bipartita; Pavona venosa; Pectinia alcicornis; Pectinia paeonea; Platygyra acuta; Platygyra pini; Platygyra sp “green”; Platygyra verweyi; Podabacia cf lanakensis; Porites annae; Porites cylindrica; Porites evermanni; Porites monticulosa; Psammocora digitata; Psammocora explanulata; Psammocora haimeana; Psammocora superficialis; Sandalolitha dentata; Seriatopora caliendrum; Stylocoeniella armata; Stylocoeniella guentheri; Stylaster sp.; Tubipora musica; Turbinaria stellulata; or any coral known in the art, or a combination thereof.

In one embodiment of this invention, the term “coral” refers to coral which is cut from a single piece of coral.

In one embodiment, coral may be machined into the described configurations, and quite complex shapes which are substantially conical, but for example, further modified to include or be shaped to include a threaded structure is envisioned and the same may be formed by appropriate machine or other processing, such as chemical processing.

In some embodiments, the solid substrate is scaled into a size/dimension so as to be most approximate to accommodate a site of desired tissue growth or repair.

In some aspects, the solid substrate is scaled into a size/dimension and of a number so as to flank or border a defect site, in some embodiments, or in other embodiments to periodically span but not fully occupy a defect site.

In some embodiments, the solid substrate comprises a hollow or hollows along a Cartesian coordinate axis of said solid substrate.

In one embodiment, a coral for use in a solid substrate of this invention comprises an average void diameter, average pore size or a combination thereof appropriate for cell seeding and/or development of vasculature.

In one embodiment, coral is washed, bleached, frozen, dried, exposed to electrical forces, magnetic forces or ultrasound waves or microwaves or electromagnetic radiation or high pressure or a combination thereof prior to use thereof.

In some embodiments, the solid substrate is of a size and/or overall shape that is appropriate for the intended purpose, as will be appreciated by the skilled artisan. For example, and in some embodiments, solid substrates for use in osteochondral therapy or repair may make use of a substrate that has a diameter of about 5-15 mm, and a height of about 5-25 mm. In some embodiments, the solid substrate has a diameter of about 1-35 mm, and a height of about 1-45 mm, or about 5-40 mm, and a height of about 5-60 mm, or about 5-15 mm, and a height of about 5-45 mm. 5-30 mm, 15-60 mm, or larger.

In some embodiments of any of the methods and uses as described herein, the hollows have a diameter ranging from about 125 to 650 mm.

In some embodiments of any of the methods and uses as described herein, the first phase has a height of between 1-7 mm.

In some embodiments of any of the methods and uses as described herein, the solid substrate has a height of about 5-40 mm.

In some embodiments of any of the methods and uses as described herein, the solid substrate has a height of about 1 cm to about 5 cm.

In some embodiments of any of the methods and uses as described herein, the solid substrate(s) is(are) placed such that the at least a second porous phase is implanted within or proximally to cartilage tissue and the at least a first porous phase is implanted within or proximally to bone tissue.

It will be appreciated by the skilled artisan that the size of the substrate may be so selected so as to be suitable to a particular application, and accordingly, this invention is not to be limited by the size of the solid substrate.

It will be appreciated by the skilled artisan that the overall shape of the substrate may be so selected so as to be suitable to a particular application, and accordingly, this invention is not to be limited by the shape of the solid substrate.

A solid substrate of this invention is characterized by a specific fluid uptake capacity value as desired for the specific application for example of at least 75%, which specific fluid uptake capacity value is determined by establishing a spontaneous fluid uptake value divided by a total fluid uptake value.

In some embodiments, the fluid is a biologic fluid, which in some embodiments, is blood, and in some embodiments, the biologic fluid is water. In some embodiments, the biologic fluid is hydrophilic. In some embodiments the fluid is a plasma or plasma-containing solution. In some embodiments, the fluid is a protein-containing or carbohydrate-containing solution. In some embodiments the fluid is a salt-containing solution. In some embodiments, the solution is a glycoprotein-containing solution.

In some embodiments, the biologic fluid is autologous with respect to a cell or tissue of a subject when said solid substrate is contacted with such cell or tissue of said subject.

In some embodiments, the biologic fluid is a blood analog as herein defined and as defined in the patent applications described herein, which are incorporated herein by reference in their entirety.

It will be appreciated by the skilled artisan that the fluid for use in determining specific fluid uptake capacity values of the solid substrates as herein described may include any appropriate described fluid, for example, Salt based solutions such as physiologic Saline (0.9% NaCl), or in some embodiments, Carbohydrate based solutions such as Glucose 1 g/L in saline, or in some embodiments, Glucose 1 g/L in WFI, or in some embodiments, Glucose 10 g/L in WFI, or in some embodiments, a Protein based solution such as BSA 50 g/L in saline, or in some embodiments, BSA 5 g/L in in WFI, or in some embodiments, BSA 0.5 g/L in in WFI, or in some embodiments, a Glycerol based solution, such as, for example, 22% Glycerol in saline, or in some embodiments, 22% Glycerol in WFI, or in some embodiments, 30% Glycerol in WFI, or in some embodiments, 44% Glycerol in WFI, or in some embodiments, a Xanthan-Gum & Glycerol solution, such as, for example, 0.025% Xanthan-Gum+30% Glycerol in WFI, or in some embodiments, combinations of the above, for example, Glycerol/Glucose/BSA/saline/Skim milk, or in some embodiments, Glucose 0.1 g/dL+BSA 5 g/dL in saline, or in some embodiments, 5g/dL skim milk in saline, or in some embodiments, 22% Glycerol+50 g/L skim milk in saline, or in some embodiments, 22% Glycerol+10 g/L Glucose in saline, or in some embodiments, 22% Glycerol+1 g/L Glucose in saline, or in some embodiments, 30% Glycerol+1 g/L Glucose in saline, or in some embodiments, 30% Glycerol+10 g/L Glucose in saline, or in some embodiments, 32.5% Glycerol+1 g/L Glucose in saline, or in some embodiments, 35% glycerol+1 g/L Glucose in saline, or in some embodiments, 35% Glycerol+1 g/L Glucose in saline, or in some embodiments, 40% Glycerol+1 g/L Glucose in saline, or in some embodiments, PEG/Tween 20/Gelatin such as, for example, 40% Glycerol+1 g/L Glucose in saline+1% PEG, or in some embodiments, 40% Glycerol+1 g/L Glucose in saline+0.1% PEG, or in some embodiments, 40% Glycerol+1 g/L Glucose in saline+0.1% PEG+0.1% Tween 20, or in some embodiments, 40% Glycerol+1 g/L Glucose in saline+0.1% PEG+0.1% Gelatin, and others, as will be appreciated by the skilled artisan.

It will also be appreciated by the skilled artisan that any such fluid for use in determining the specific fluid uptake capacity values of the solid substrates as herein described may also be considered to represent an envisioned “blood analogue” as herein described.

In some embodiments, the process further comprises the step of contacting the material with a fluid for from 2-15 minutes to promote spontaneous fluid uptake of said fluid within said coralline-based solid material to arrive at said spontaneous fluid uptake value. In some embodiments, the process may allow for the contacting of the material with a fluid for from 0.5-15 minutes, or in some embodiments, from 0.5-5 minutes, or in some embodiments, 10-60 minutes, or in some embodiments, from 60 to 90 minutes, or in some embodiments, other intervals, to promote spontaneous fluid uptake. The skilled artisan will appreciate that the amount of time for which the fluid is applied to determine the spontaneous uptake may be extended or shortened as a function of the dimensions and geometry of the sample substrate being assessed. In some embodiments, when a larger sample is being assessed, the process further comprises the step of contacting the material with a fluid for from 2-24 hours to promote spontaneous fluid uptake of said fluid within said coralline-based solid material to arrive at said spontaneous fluid uptake value

In some embodiments, the process further comprises the step of contacting said solid material with a fluid and applying negative pressure to the solid implant material to promote maximal uptake of said fluid within said coralline-based solid material to arrive at said total fluid uptake value. In some embodiments, application of positive pressure is via the application of a vacuum to the substrate immersed in the fluid, promoting entry of the fluid therewithin.

In some embodiments, the process may further comprise the step of contacting the solid implant material with a fluid and applying positive pressure to same to promote maximal uptake of fluid within the solid implant material to arrive at said total fluid uptake value. According to this aspect, and in some embodiments, care will be taken to ensure that the application of pressure does not in any way compromise the structural integrity of the solid substrate.

In some embodiments, application of positive pressure is via any manual means, for example, via the use of any applicator, syringe, etc., gravitational pressure, and others, as will be appreciated by the skilled artisan. In some embodiments, application of positive pressure is via forced osmosis, centrifugation and others. In some embodiments, combinations of the described methods and others are envisioned.

In some embodiments, the solid substrate for promoting cell or tissue growth or restored function comprises a coralline or coralline derivative, or other appropriate solid implant material characterized by having a contact angle value of less than 60 degrees, when in contact with a fluid.

Methods for determining a contact angle are well known, and any appropriate method can be used.

In some aspects, the sample is further dried under vacuum and/or heated or pressurized or steam treated.

In some embodiments, for aspects relating to a specific fluid uptake capacity value, such value is a function of change in weight in the solid implant material.

According to this aspect and in some embodiments, the dry weight for each sample is recorded and fluid as described herein is added an assay container.

According to this aspect and in some embodiments, at least 1:1 ratio of the size of the sample in mm to the volume of fluid added in ml is applied to the container. In some embodiments, the amount of fluid applied is in excess, as compared to the sample size.

According to this aspect and in some embodiments, once the initial fluid uptake is assessed, according to this aspect and in some embodiments, the solid substrate sample is then brought into contact with the fluid and the weight of the solid substrate sample is assessed. In other embodiments the specific gravity is assessed by gradient centrifugation of by the Archimedean principle.

According to this aspect and in some embodiments, spontaneous fluid uptake is assessed and a spontaneous fluid uptake value is established, based on the change in weight of the sample.

According to this aspect and in some embodiments, the specific fluid uptake capacity value is a function of change in fluid volume of applied fluid to said marine organism skeletal derivative-based solid material. According to this aspect, spontaneous fluid uptake is assessed and a spontaneous fluid uptake value is established based on the complete uptake of the volume applied to the sample.

According to this aspect and in some embodiments, the process then further comprises contacting a significantly increased amount of fluid with the sample and applying pressure thereto to promote maximal fluid uptake to the total fluid uptake capacity of the sample.

According to this aspect and in some embodiments, as noted, such pressure may be either positive or negative pressure, and the application time is for a period of time sufficient to ensure maximal uptake of the applied fluid into the marine organism skeletal derivative sample.

According to this aspect and in some embodiments, such time may include an interval of from 0.5-60 minutes, or in some embodiments, when a larger sample is being assessed, such time may include an interval of from 2-24 hours to arrive at said spontaneous fluid uptake value. It will be appreciated that the time intervals recited herein are applicable for any embodiment with regard thereto as described herein. The skilled artisan will appreciate that the amount of time for which the fluid is applied to determine the full capacity fluid uptake may be extended or shortened as a function of the dimensions and geometry of the sample substrate being assessed.

According to these aspects, the total fluid uptake capacity is thus assessed and the specific fluid uptake capacity value is then determined.

In some embodiments, when the sample is utilized in vivo in subsequent applications, in some aspects, the sample is first contacted with autologous biological fluids or materials from the host prior to implantation into the same, verifying the observed enhanced fluid uptake phenotype as herein described.

In one embodiment of this invention, the solid substrate may further comprise an additional material.

In some embodiments, such additional material may include a polymer.

The term “polymer” refers, in some embodiments, to the presence of a layer of polymeric material in association with at least a portion of the solid substrate material. In some embodiments, such polymer layer is a coating for the solid substrate material.

In some embodiments, such coating may be over the entirety of the solid substrate, and in some embodiments, such coating may penetrate to within the voids and/or pores and/or hollows of the solid substrate. In some embodiments, such coating may be selectively applied to a particular region of the solid substrate, such that it creates a separate phase on the solid substrate, and in some embodiments, such polymer may be so applied that a thick polymer layer or phase is associated with a portion of a solid substrate, thereby creating a separate polymer phase in association with the solid substrate as herein described.

In one embodiment, the polymer coating provides added features to the solid substrates as herein described, for example, added tensile strength, added flexibility, reduced brittleness, and other attributes, to the solid substrate and in some embodiments, the polymer coating results in greater cellular attraction and attachment to the solid substrates as herein described, which in turn, inter alia, results in enhanced repair in terms of quantity, quality and timing of repair. In some embodiments, the polymer coating enhance cells proliferation and/or differentiation into desired mature tissue which in turn, inter alia, results in enhanced repair in terms of quantity, quality and timing of repair.

In one embodiment of this invention, a polymer coating is permeable. In one embodiment, the permeable polymer coating comprises a special porous membrane. In one embodiment, the term “permeable” refers to having pores and openings. In one embodiment, the permeable polymer coating of this invention has pores and openings which allow entry of nutrients, a therapeutic compound, a cell population, a chelator, or a combination thereof. In one embodiment, the permeable polymer coating of this invention has pores and openings which allow exit/release of nutrients, a therapeutic compound, a cell population, a chelator, or a combination thereof.

In one embodiment, a polymer coating of this invention is discontinuous. In one embodiment, a region or a plurality of sub-regions of the coral of this invention comprise an absence of polymer coating, allowing direct contact between the coral and the environment.

In some embodiments, the solid substrate incorporates a biocompatible polymer therewithin, which is associated with the aragonite or calcite component, via any physical or chemical association. In some embodiments, the polymer is a part of a hydrogel, which is incorporated in the solid substrates of this invention. In some embodiments, such hydrogel-containing solid substrates may thereafter be lyophilized or dessicated, and may thereafter be reconstituted.

In some embodiments of the solid substrates of this invention, the polymer may be applied to the solid substrate so as to form a separate phase, or in some embodiments, the polymer may be applied as a layer onto the solid substrate, or in some embodiments, the solid substrate may comprise both polymer as an internal or externally associated layer with a separate phase attached thereto comprising the same or a different polymeric material.

Such polymer-containing solid substrates may be particularly suited for cartilage repair, regeneration or enhancement of formation thereof. In some embodiments, according to this aspect, for example, in the treatment of osteochondral defects, the solid substrate is of a dimension suitable for incorporation within affected bone, and further comprises a polymer-containing phase, which phase, when inserted within the affected defect site, is proximal to affected cartilage. In another aspect and representing an embodiment of this invention, the solid substrate comprises a polymer, which has permeated within the voids and pores of the solid substrate, which solid substrate is inserted within a site of cartilage repair and which polymer facilitates cartilage growth, regeneration or healing of the defect site.

Such polymer-containing solid substrates may be particularly suited for bone repair, regeneration or enhancement of formation thereof. In some embodiments, according to this aspect, for example, in the treatment of bone edema, bone breakage or fragmentation, disease or defect, the coralline-based solid substrate is of a dimension suitable for incorporation within affected bone, and further comprises a polymer, which polymer has permeated within the voids and pores of the solid substrate, which solid substrate is inserted within the bone and which polymer facilitates bone growth, regeneration or healing of the defect site.

In one embodiment, a polymer coating of this invention comprises a natural polymer comprising, collagen, fibrin, elastin, silk, hyaluronic acid, sodium hyaluronate, cross linked hyalronic acid, chitosan, cross linked chitosan, alginate, calcium alginate, cross linked calcium alginate and any combinations thereof.

In one embodiment, the polymer comprises synthetically modified natural polymers, and may include cellulose derivatives such as alkyl celluloses, hydroxyalkyl celluloses, cellulose ethers, cellulose esters and nitrocelluloses. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate and cellulose sulfate sodium salt.

In one embodiment, of this invention, a polymer comprises a synthetic biodegradable polymer. In one embodiment of this invention, a synthetic biodegradable polymer comprises alpha-hydroxy acids including poly-lactic acid, polyglycolic acid, enantioners thereof, co-polymers thereof, polyorthoesters, and combinations thereof.

In one embodiment, a polymer of this invention comprises a poly(cianoacrylate), poly(alkyl-cianoacrylate), poly(ketal), poly(caprolactone), poly(acetal), poly(α-hydroxy-ester), poly(a-hydroxy-ester), poly(hydroxyl-alkanoate), poly(propylene-fumarate), poly (imino-carbonate), poly(ester), poly(ethers), poly(carbonates), poly(amide), poly(siloxane), poly(silane), poly(sulfide), poly(imides), poly(urea), poly(amide-enamine), poly(organic acid), poly(electrolytes), poly(p-dioxanone), poly(olefin), poloxamer, inorganic or organomatallic polymers, elastomer, or any of their derivatives, or a copolymer obtained by a combination thereof.

In one embodiment, a polymer of this invention comprises poly(D,L-lactide-co-glycolide) (PLGA). In another embodiment, the polymer comprises poly(D,L-lactide) (PLA). In another embodiment, the polymer comprises poly(D,L-glycolide) (PGA). In one embodiment, the polymer comprises a glycosaminoglycan.

In one embodiment, the polymer comprises synthetic degradable polymers, which may include, but are not limited to polyhydroxy acids, such as poly(lactide)s, poly(glycolide)s and copolymers thereof; poly(ethylene terephthalate); poly(hydroxybutyric acid); poly(hydroxyvaleric acid); poly[lactide-co-(ε-caprolactone)]; poly[glycolide-co(ε-caprolactone)]; poly(carbonate)s, poly(pseudo amino acids); poly(amino acids); poly(hydroxyalkanoate)s; poly(anhydrides); poly(ortho ester)s; and blends and copolymers thereof.

In one embodiment of this invention, a polymer comprises proteins such as zein, modified zein, casein, gelatin, gluten, serum albumin, collagen, actin, α-fetoprotein, globulin, macroglobulin, cohesin, laminin, fibronectin, fibrinogen, osteocalcin, osteopontin, osteoprotegerin, or others, as will be appreciated by one skilled in the art. In another embodiment, a polymer may comprise cyclic sugars, cyclodextrins, synthetic derivatives of cyclodextrins, glycolipids, glycosaminoglycans, oligosaccharide, polysaccharides such as alginate, carrageenan (χ, λ, μ, κ), chitosane, celluloses, condroitin sulfate, curdlan, dextrans, elsinan, furcellran, galactomannan, gellan, glycogen, arabic gum, hemicellulose, inulin, karaya gum, levan, pectin, pollulan, pullulane, prophyran, scleroglucan, starch, tragacanth gum, welan, xanthan, xylan, xyloglucan, hyaluronic acid, chitin, or a poly(3-hydroxyalkanoate)s, such as poly(β-hydroxybutyrate), poly(3-hydroxyoctanoate) or poly(3-hydroxyfatty acids), or any combination thereof.

In one embodiment, the polymer comprises a bioerodible polymer such as poly(lactide-co-glycolide)s, poly(anhydride)s, and poly(orthoester)s, which have carboxylic groups exposed on the external surface as the smooth surface of the polymer erodes, which may also be used. In one embodiment, the polymer contains labile bonds, such as polyanhydrides and polyesters.

In one embodiment, a polymer may comprise chemical derivatives thereof (substitutions, additions, and elimination of chemical groups, for example, alkyl, alkylene, hydroxylations, oxidations, and other modifications routinely made by those skilled in the art), blends of, e.g. proteins or carbohydrates alone or in combination with synthetic polymers.

In one embodiment of this invention, the polymer is biodegradable. In one embodiment, the term “biodegradable” or grammatical forms thereof, refers to a material of this invention, which is degraded in the biological environment of the subject in which it is found. In one embodiment, the biodegradable material undergoes degradation, during which, acidic products, or in another embodiment, basic products are released. In one embodiment, bio-degradation involves the degradation of a material into its component subunits, via, for example, digestion, by a biochemical process. In one embodiment, biodegradation may involve cleavage of bonds (whether covalent or otherwise), for example in a polymer backbone of this invention. In another embodiment, biodegradation may involve cleavage of a bond (whether covalent or otherwise) internal to a side-chain or one that connects a side chain to, for example a polymer backbone.

In one embodiment, the polymer as applied to the solid substrates of this invention has a thickness of between 2.0 μm and 0.1 μm. In one embodiment, the polymer coating has a thickness of about 1.0 μm. In one embodiment, the polymer coating of this invention has a thickness of between 10 μm and 50 μm. In one embodiment, the polymer coating as applied to the solid substrates of this invention has a thickness of about 10-25, or about 15-30, or about 25-50 μm. In one embodiment, the polymer coating as applied to the solid substrates of this invention has a thickness of about 0.0001-0.1 μm. In one embodiment, the polymer coating as applied to the solid substrates of this invention has a thickness of about 20-200 μm. In one embodiment, the polymer coating as applied to the solid substrates of this invention has a thickness of about 100-1500 μm.

In some embodiments, the polymer as applied to the solid substrates of this invention is a thin coating, which is associated with the solid substrates of this invention and has a thickness as indicated hereinabove.

In some embodiments, the polymer as applied to the solid substrates of this invention is applied throughout the solid substrates of this invention, such that, in some embodiments, the pores and voids within the solid substrates of the invention may be filled with polymers as herein described, and such polymer layer as applied may have a thickness of about 60-900 μm.

In some embodiments, the polymer is applied to an apical surface of an implant, in situ, as part of an implantation procedure of this invention.

In some embodiments, the methods and/or uses as described herein may make use of a kit comprising a solid substrate as herein described, which in some embodiments may optionally comprise a separate container comprising a fluid as herein described, a biocompatible polymer as herein described, and tools for application to and implantation of the solid substrate.

In one embodiment, the solid substrates of this invention may further comprise an effector compound, which in some embodiments, may be associated directly with the solid substrates of this invention, or in some embodiments, may be associated with a polymer, and applied in connection therewith.

In one embodiment of this invention, the effector compound comprises a cytokine, a bone morphogenetic protein (BMP), growth factors, a chelator, a cell population, a therapeutic compound, or an antibiotic, or any combination thereof.

In one embodiment of this invention, the phrase “a therapeutic compound” refers to a peptide, a protein or a nucleic acid, or a combination thereof. In another embodiment, the therapeutic compound is an antibacterial, antiviral, antifungal or antiparasitic compound. In another embodiment, the therapeutic compound has cytotoxic or anti-cancer activity. In another embodiment, the therapeutic compound is an enzyme, a receptor, a channel protein, a hormone, a cytokine or a growth factor. In another embodiment, the therapeutic compound is immunostimulatory. In another embodiment, the therapeutic compound inhibits inflammatory or immune responses. In one embodiment, the therapeutic compound comprises a pro-angiogenic factor.

In one embodiment, the effector compound comprises, an anti-helminth, an antihistamine, an immunomodulatory, an anticoagulant, a surfactant, an antibody, a beta-adrenergic receptor inhibitor, a calcium channel blocker, an ace inhibitor, a growth factor, a hormone, a DNA, an siRNA, or a vector or any combination thereof.

In one embodiment, the phrase “effector compound” refers to any agent or compound, which has a specific purpose or application which is useful in the treatment, prevention, inhibition, suppression, delay or reduction of incidence of infection, a disease, a disorder, or a condition, when applied to the solid substrates, kits and/or methods of this invention. An effector compound of this invention, in one embodiment, will produce a desired effect which is exclusive to the ability to image the compound. In some embodiments, the effector compound may be useful in imaging a site at which the compound is present, however, such ability is secondary to the purpose or choice of use of the compound.

In one embodiment of this invention, term “effector compound” is to be understood to include the terms “drug” and “agent”, as well, when referred to herein, and represents a molecule whose incorporation within the solid substrate and/or kits of this invention, or whose use thereof, is desired. In one embodiment, the agent is incorporated directly within a solid substrate, and/or kit of this invention. In another embodiment, the agent is incorporated within a solid substrate and/or kit of this invention, either by physical interaction with a polymer coating, a coral, or coral particles of this invention, and/or a kit of this invention, or association thereto.

In one embodiment, the “effector compound” is a therapeutic compound.

In one embodiment, the phrase “a therapeutic compound”, refers to a molecule, which when provided to a subject in need, provides a beneficial effect. In some cases, the molecule is therapeutic in that it functions to replace an absence or diminished presence of such a molecule in a subject. In one embodiment, the molecule is a nucleic acid coding for the expression of a protein is absent, such as in cases of an endogenous null mutant being compensated for by expression of the foreign protein. In other embodiments, the endogenous protein is mutated, and produces a non-functional protein, compensated for by the expression of a heterologous functional protein. In other embodiments, expression of a heterologous protein is additive to low endogenous levels, resulting in cumulative enhanced expression of a given protein. In other embodiments, the molecule stimulates a signaling cascade that provides for expression, or secretion, or others of a critical element for cellular or host functioning.

In another embodiment, the therapeutic compound may be natural or non-natural insulins, amylases, proteases, lipases, kinases, phosphatases, glycosyl transferases, trypsinogen, chymotrypsinogen, carboxypeptidases, hormones, ribonucleases, deoxyribonucleases, triacylglycerol lipase, phospholipase A2, elastases, amylases, blood clotting factors, UDP glucuronyl transferases, ornithine transcarbamoylases, cytochrome p450 enzymes, adenosine deaminases, serum thymic factors, thymic humoral factors, thymopoietins, growth hormones, somatomedins, costimulatory factors, antibodies, colony stimulating factors, erythropoietin, epidermal growth factors, hepatic erythropoietic factors (hepatopoietin), liver-cell growth factors, interleukins, interferons, negative growth factors, fibroblast growth factors, transforming growth factors of the a family, transforming growth factors of the β family, gastrins, secretins, cholecystokinins, somatostatins, serotonins, substance P, transcription factors or combinations thereof.

In any of the embodiments herein, solid substrates, and their use in the methods of the present invention may further comprise, or be implanted with, other compounds such as, for example, antioxidants, growth factors, cytokines, antibiotics, anti-inflammatoires, immunosuppressors, preservative, pain medication, other therapeutics, and excipient agents. In one embodiment, examples of growth factors that may be administered in addition to the HMG-COA reductase inhibitor include, but are not limited to, epidermal growth factor (EGF), transforming growth factor-alpha (TGF-α), transforming growth factor-beta (TGF-β), human endothelial cell growth factor (ECGF), granulocyte macrophage colony stimulating factor (GM-CSF), bone morphogenetic protein (BMP), nerve growth factor (NGF), vascular endothelial growth factor (VEGF), fibroblast growth factor (FGF), insulin-like growth factor (IGF), cartilage derived morphogenetic protein (CDMP), platelet derived growth factor (PDGF), or any combinations thereof. Examples of antibiotics include antimicrobials and antibacterials.

In any of the embodiments herein, solid substrates, and their use in the methods of the present invention may further comprise, or be implanted with, plasma, platelet rich plasma, any growth factor as appropriate, any glycosaminoglycan, in particular, hyaluronic acid and any useful form of same, or any combination of same.

In one embodiment, effector compounds for use in a solid substrate and/or a kit of this invention and/or a method of this invention may comprise, inter-alia, an antibody or antibody fragment, a peptide, an oligonucleotide, a ligand for a biological target, an immunoconjugate, a chemomimetic functional group, a glycolipid, a labelling agent, an enzyme, a metal ion chelate, an enzyme cofactor, a cytotoxic compound, a bactericidal compound, a bacteriostatic compound, a fungicidal compound, a fungistatic compound, a chemotherapeutic, a growth factor, a hormone, a cytokine, a toxin, a prodrug, an antimetabolite, a microtubule inhibitor, a radioactive material, or a targeting moiety, or any combination thereof.

In one embodiment, the solid substrates and/or kits of this invention and/or methods of this invention comprise or make use of an oligonucleotide, a nucleic acid, or a vector. In some embodiments, the term “oligonucleotide” is interchangeable with the term “nucleic acid”, and may refer to a molecule, which may include, but is not limited to, prokaryotic sequences, eukaryotic mRNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

The solid substrates and/or kits of this invention and/or methods of use of this invention may comprise nucleic acids, in one embodiment, or in another embodiment, the solid substrates and/or kits of this invention and/or methods of use of this invention may include delivery of the same, as a part of a particular vector. In one embodiment, polynucleotide segments encoding sequences of interest can be ligated into commercially available expression vector systems suitable for transducing/transforming mammalian cells and for directing the expression of recombinant products within the transduced cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

In one embodiment, effector compounds for use in a solid substrate and/or a kit of this invention and/or a method of this invention may comprise, inter-alia, a cytokine, a bone morphogenetic protein (BMP), growth factor, a chelator, a cell population, a therapeutic compound, an anti-inflammatory compound, a pro-angiogenic compound or an antibiotic, or any combination thereof.

It will be appreciated that the solid substrates as herein described, and including any embodied addition to/incorporation within same, refers to such substrates possessing tapered sides as herein described, or in some embodiments, specifically shaped to be substantially ovoid in shape and optionally further comprising a taper, as described herein.

In some embodiments, the solid substrates of this invention may be seeded with cells, cell populations or tissue.

In some embodiments, the cells or tissue comprise stem or progenitor cells, or a combination thereof.

It will be appreciated that any appropriate stem or progenitor cell, from any source or obtained via any protocol is envisioned.

In some embodiments, adipose tissue derived stem cells are specifically envisioned for use in the methods of this invention and for incorporation with the solid substrates of this invention or kits of this invention.

In one embodiment of this invention, the cells or tissue as used in accordance with the substrates, methods of use or kits of this invention, are engineered to express a desired product.

In one embodiment, the phrase “a cell population” refers to a transfected cell population, a transduced cell population, a transformed cell population, or a cell population isolated from a subject, or a combination thereof. In some embodiments, transfected, transduced or transformed cells, may be seeded on the solid substrate, or in some embodiments, may be incorporated into a polymeric application thereto, or a combination thereof.

In one embodiment, a cell population of this invention comprises mesenchymal stem cells. In one embodiment, the mesenchymal stem cells are transformed.

In one embodiment, a cell population comprises cells beneficial in repair of a tissue for which the implantation of a solid substrate of this invention is desired.

In some embodiments, the cells are beneficial in and/or promote cartilage and/or bone formation and/or repair. Such cells may include chondroblasts or chondrocytes; fibrochondrocyte; osteocyte; osteoblast; osteoclast; synoviocyte; bone marrow cell; stromal cell; stem cell; embryonic stem cell; precursor cell, derived from adipose tissue; peripheral blood progenitor cell; stem cell isolated from adult tissue; genetically transformed cell; or a combination thereof. In another embodiment, a precursor cell may refer to a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells. The precursor cells for use in the method of the present invention are prepared from an organ tissue of the recipient mammal (i.e. autologous), or a syngeneic mammal. In another embodiment, allogeneic and xenogeneic precursor cells may be utilized.

In one embodiment, the solid substrate of this invention incorporates any cell which may participate in tissue repair, for example, in cartilage and/or bone formation or repair. In some embodiments, such cells represent autografts, in that cells are cultured ex-vivo to seed the cells on the solid substrates of the invention, and such seeded solid substrates are implanted into the subject.

In some embodiments, such cells may represent allografts or xenografts, which may be incorporated within the solid substrates of this invention and implanted within a site of repair.

In one embodiment, a coral of this invention comprises a cell population from in vitro culture of the coral for a time period sufficient to seed the cells in the coral. In one embodiment, the cell population is a mesenchymal stem cell population, chondrocyte; fibrochondrocyte; osteocyte; osteoblast; osteoclast; synoviocyte; bone marrow cell; stromal cell; stem cell; embryonic stem cell; precursor cell, derived from adipose tissue; peripheral blood progenitor cell; stem cell isolated from adult tissue; genetically transformed cell; or a combination thereof. In one embodiment, the mesenchymal stem cells; chondrocyte; fibrochondrocyte; osteocyte; osteoblast; osteoclast; synoviocyte; bone marrow cell; stromal cell; stem cell; embryonic stem cell; precursor cell, derived from adipose tissue; peripheral blood progenitor cell; stem cell isolated from adult tissue; genetically transformed cell; or a combination thereof seeded in vitro are transformed. In one embodiment, the cell population comprises a cell population beneficial for cartilage repair. In one embodiment, the culture comprises a chelator. In one embodiment of this invention, the chelator in a culture comprises a calcium chelator.

In some embodiments, the solid substrate may further serve as a bone substitute or bone void filler. In some embodiments, the solid substrate may further incorporate a bone-substitute or bone void filler. In some embodiments, such bone-containing material may comprise autlogous or allogeneic bone. In some embodiments, s bone-containing material may comprise animal bone.

In some embodiments, solid substrates of this invention may be applied for use in a subject with a bone defect in need of repair, wherein access to the bone defect results in the creation of a defect in the overlying cartilage, and the solid substrates of this invention allow for ideal healing of affected bone or bone and cartilage tissues.

In other embodiments, such solid substrates may be administered to a subject with a cartilage defect in need of repair, wherein optimal insertion of the solid substrate for stimulation of cartilage repair necessitates anchoring of the scaffold in the underlying bone, for example, by creating a minimal void in the underlying bone for insertion of the solid substrates, and once inserted, the solid substrate facilitates repair of both the overlying cartilage and underlying bone.

In other embodiments, such solid substrate may be administered to a subject with an osteochondral defect, where both bone and cartilage tissue are in need of repair as part of the pathogenesis of the disorder. The solid substrates according to this aspect are, in some embodiments, particularly suited for such applications.

It will be appreciated by the skilled artisan, that the applications, in particular, as related to bone therapy may include use of a solid substrate that incorporates any additional element as described herein, including, for example, bone allograft, bone autograft, bone substitutes, known bone void fillers, therapeutic compounds, and the like.

In some embodiments, the methods/uses/kits/materials of this invention as described herein are for use in a subject who suffers from a full or partial thickness articular cartilage defect; osteochondral defect; osteoarthritis, a joint defect or a defect resulting from trauma, sports, or repetitive stress. In some embodiments, according to this aspect, the suffers from a bone fracture, bone defect, bone edema, osteoporosis, or a defect resulting from trauma, sports, or repetitive stress.

In some embodiments, the methods/uses/kits/materials of this invention as described herein may find application in resurfacing an affected joint in a subject, as will be known to the skilled artisan.

This invention provides, in some embodiments, solid substrates for use in repairing cartilage and/or bone tissue defects associated with physical trauma, or cartilage and/or bone tissue defects associated with a disease or disorder in a subject.

In some aspects, it is particularly contemplated that the methods, solid substrates, kits and tools and systems of the invention are suitable for hip replacement, great toe fusion, arthrodesis, ankle replacement or fusion, total or partial knee replacement procedures, including any or all of same.

In some embodiments, multiple coralline solid substrates as herein described are inserted to maximally occupy a defect site, to accommodate proper insertion into the desired region within a desired implantation site.

In one embodiment, the phrase “cartilage repair” refers to restoring a cartilage defect to a more healthful state. In one embodiment, restoring cartilage results in regeneration of cartilage tissue. In one embodiment, restoring cartilage results in regeneration of a full or partial thickness articular cartilage defect. In one embodiment, restoring cartilage results in complete or partial regeneration of cartilage tissue at a site of cartilage repair. In one embodiment, cartilage repair may result in restoration/repair of missing or defective bone tissue, wherein repair of a cartilage defect necessitates removal of bone tissue at a site of cartilage repair. In one embodiment, restoring cartilage results in regeneration of osteochondral defect. In one embodiment, cartilage repair comprises restoring cartilage defects of joints (e.g. knee, elbow, ankle, toe, finger, hip, shoulder joints), of ears, of a nose, or of a wind pipe.

In one embodiment, the phrase “bone repair” refers to restoring a bone defect to a more healthful state. In one embodiment, restoring bone results in regeneration of bone tissue. In one embodiment, restoring bone results in the filling in of any fracture or void within a bone tissue. In one embodiment, restoring bone results in complete or partial regeneration of bone tissue at a site of bone repair. In one embodiment, bone repair may result in restoration/repair of missing or defective bone tissue. In one embodiment, bone repair comprises restoring bone defects of any bone, treating bone edema, and other bone disorders, as needed.

In some embodiments, the phrase “bone repair” refers to the treatment of a subject with osteoporosis, Paget's disease, fibrous dysplasias, bone edema or osteodystrophies. In another embodiment, the subject has bone and/or cartilage infirmity. In another embodiment, the subject has other bone remodeling disorders include osteomalacia, rickets, rheumatoid arthritis, achondroplasia, osteochodrytis, hyperparathyroidism, osteogenesis imperfecta, congenital hypophosphatasia, fribromatous lesions, multiple myeloma, abnormal bone turnover, osteolytic bone disease, periodontal disease, or a combination thereof. In one embodiment, bone remodeling disorders include metabolic bone diseases which are characterized by disturbances in the organic matrix, bone mineralization, bone remodeling, endocrine, nutritional and other factors which regulate skeletal and mineral homeostasis, or a combination thereof. Such disorders may be hereditary or acquired and in one embodiment, are systemic and affect the entire skeletal system.

In other aspects, the invention specifically contemplates use of the solid substrates as herein described and methods for use of same for treating a bone and/or cartilage defect arising as a consequence of tumor or avascular necrosis.

The solid substrates, kits and methods of the invention may also be used to enhance bone and/or cartilage formation in conditions where a bone and/or cartilage deficit is caused by factors other than bone remodeling disorders. Such bone deficits include fractures, bone trauma, conditions associated with post-traumatic bone surgery, post-prosthetic joint surgery, post plastic bone surgery, bone chemotherapy, post dental surgery and bone radiotherapy. Fractures include all types of microscopic and macroscopic fractures. In one embodiment, some examples of fractures includes avulsion fracture, comminuted fracture, transverse fracture, oblique fracture, spiral fracture, segmental fracture, displaced fracture, impacted fracture, greenstick fracture, torus fracture, fatigue fracture, intraarticular fracture (epiphyseal fracture), closed fracture (simple fracture), open fracture (compound fracture) and occult fracture. In one embodiment, fractures meant to be treated using the methods of the present invention are non-union fractures.

In one embodiment, methods, materials and kits of this invention are utilized for induced or enhanced repair of a cartilage and/or bone defect or disorder or disease. In one embodiment, the cartilage defect results from a trauma, a tear, a sports injury, a full thickness articular cartilage defect, a joint defect, or a repetitive stresses injury (e.g., osteochondral fracture, secondary damage due to cruciate ligament injury). In one embodiment, the cartilage disorder comprises a disease of the cartilage. In one embodiment, methods of this invention induce or enhance cartilage repair in osteoarthritis, rheumatoid arthritis, aseptic necrosis, osteoarthritis, including osteochondritis dissecans, articular cartilage injuries, chondromalacia patella, chondrosarcoma, chondrosarcoma—head and neck, costochondritis, enchondroma, hallux rigidus, hip labral tear, osteochondritis dissecans, torn meniscus, relapsing polychondritis, canine arthritis, fourth branchial arch defect or cauliflower ear. In one embodiment, methods of this invention induce or enhance cartilage repair in degenerative cartilagenous disorders comprising disorders characterized, at least in part, by degeneration or metabolic derangement of connective tissues of the body, including not only the joints or related structures, including muscles, bursae (synovial membrane), tendons, and fibrous tissue, but also the growth plate, meniscal system, and intervertebral discs.

In one embodiment, methods, materials and kits of this invention are utilized for resurfacing joints and in some embodiments, the methods, materials and kits of this invention in use as described herein, prevent, reduce the need, delay the need or abrogate the need for joint replacement.

In one embodiment, the solid substrates, kits and methods of the invention may also be used to augment long bone fracture repair; generate bone in segmental defects; provide a bone graft substitute for fractures; facilitate tumor reconstruction or spine fusion; provide a local treatment (by injection) for weak or osteoporotic bone, such as in osteoporosis of the hip, vertebrae, or wrist, or a combination thereof. In another embodiment, the solid substrates, kits and methods of the invention may also be used in a method to accelerate the repair of fractured long bones; treat of delayed union or non-unions of long bone fractures or pseudoarthrosis of spine fusions; induce new bone formation in avascular necrosis of the hip or knee, or a combination thereof.

In some embodiments, the solid substrates, kits and methods of the invention may also be used as an alternative to joint replacement, for any bone as herein described, e.g. hip, knee, shoulder, elbow, ankle, and others as will be appreciated by the skilled artisan.

In one embodiment, methods of this invention are evaluated by examining the site of cartilage and/or bone tissue repair, wherein assessment is by histology, histochemistry, palpation, biopsy, endoscopy, arthroscopy, or imaging techniques comprising X-ray photographs, computerized X-ray densitometry, computerized fluorescence densitometry, CT, MRI or another method known in the art, or any combination thereof.

In one embodiment, a method of this invention comprises inducing and enhancing cartilage and/or bone repair wherein implanting a solid substrate of this invention within a site of cartilage and/or bone repair influences and improves cartilage and/or bone repair and optionally using the specialized tools of this invention.

In one embodiment, a method of this invention induces or enhances cartilage and/or bone repair, wherein the solid substrate attracts a population of cells to the solid substrate, thereby influencing or improving cartilage and/or bone repair.

A clinician skilled in the art will recognize that methods of this invention, which entail implanting a coralline solid substrate within a site of cartilage and/or bone repair, may require preparation of a site of cartilage and/or bone repair. These preparations may occur prior to implantation of a coralline solid substrate or simultaneously with implantation. For example, cartilage and/or bone tissue and/or other tissues proximal to a site of cartilage and/or bone repair may initially be drilled through to create a channel of dimensions appropriate for a coralline solid substrate used in the methods of this invention. Then the coralline solid substrate is implanted within the site so that a region of the coralline solid substrate penetrates the drilled cartilage and/or bone tissues. Alternatively, the coralline solid substrate may be attached to a tool capable of penetrating through cartilage and/or bone or other tissues, or a combination thereof. In this case, as the tool penetrates through the cartilage and/or bone tissue, the attached coralline solid substrate is simultaneously implanted.

In some embodiments, following implantation of the solid substrate within a repair site, or several solid substrates within the repair site, the solid substrate is processed to optimize incorporation and optimal cartilage and/or bone repair. In some embodiments, such processing may comprise cutting, sanding or otherwise smoothing the surface of the solid substrate or coralline solid substrates, for optimal repair.

It will be apparent to those skilled in the art that various modifications and variations can be made in the solid substrates, kits, process and methods of the present invention without departing from the spirit or scope of the invention.

In some embodiments, the methods of this invention further comprise matching an optimized solid substrate in terms of its angle of taper to the angle of the tapered sides of the tissue shaper and alignment tool used in some embodiments of the methods of this invention.

In some embodiments, the methods of this invention include any method for tissue repair making use of a solid substrate or tool as herein described.

This invention specifically contemplates customized applications, wherein a solid substrate for implantation is specifically prepared in a customized manner to best fit a defect site in a subject in need of implantation of same.

In some aspects, this invention specifically contemplates that customization, in particular, with respect to implantation procedures within a curved tissue site in a subject include idealized preparation of a solid substrate for implantation, for example, via compiling information from a variety of sources such as MRI and/or CT scans, such that a plurality of medical images of a bone region with a defect area are obtained and converted into three-dimensional data.

In some aspects, such three-dimensional data in turn is used via automated systems to specifically machine an appropriate and idealized implant.

In some embodiments, such three-dimensional data in turn is used to facilitate selection of an implant from a variety of standard implants of varying dimensions and topographies, to promote selection of a best choice for implant from among a series of available implants.

In some embodiments, in either case, whereby a truly optimized implant is specifically and in a custom manner machined to ideally fit a subject, or an optimized implant reflective of a best fit from a wide variety of standards is chosen, the implant may further contain tapered sides as herein described and/or a rounded surface, as herein described.

In some aspects, this invention specifically contemplates that customization, in particular, with respect to implantation procedures within a curved tissue site in a subject include idealized preparation of a tool as herein described for implantation of the optimally selected, or fully customized implant, for example, via compiling information from a variety of sources such as MRI and/or CT scans, such that a plurality of medical images of a bone and/or cartilage region with a defect area are obtained and converted into three-dimensional data and tools for use in implantation in such defect site are therefore machined and prepared in a fully customized manner.

In some aspects, such three-dimensional data in turn is used via automated systems to specifically machine an appropriate and idealized tool.

In some embodiments, such three-dimensional data in turn is used to facilitate selection of an optimal tool from a variety of standard tools of varying dimensions and topographies, to promote selection of a best choice for implantation tools from among a series of available such tools.

In some embodiments, in either case, whereby a truly optimized tool is specifically selected and in a custom manner machined to ideally fit a subject, or an optimized tool reflective of a best fit from a wide variety of standards is chosen, the tools may further contain adaptations to specifically accommodate tapered sides of the implant as herein described and/or a rounded surface, as herein described.

Such substrate and tool custom preparation and/or selection allows for ideal design of a solid substrate and tool for implanting same in terms of its dimensions and for example, including a taper as herein described and/or an appropriate radius of curvature as herein described, and fabrication of same.

In some embodiments, the tools of this invention, in particular tools comprising a radius of curvature, as herein described are also fabricated via known methods to specifically provide a best fit for the subject tissue being manipulated in a highly customized manner.

In some embodiments, the term “comprise” or grammatical forms thereof, refers to the inclusion of the indicated components of this invention, as well as inclusion of other active agents, and pharmaceutically acceptable carriers, excipients, emollients, stabilizers, etc., as are known in the pharmaceutical industry.

In one embodiment, the term “about” refers to a variance of from 1-10%, or in another embodiment, 5-15%, or in another embodiment, up to 10%, or in another embodiment, up to 25% variance from the indicated values, except where context indicates that the variance should not result in a value exceeding 100%.

In one embodiment, the present invention provides combined preparations. In one embodiment, the term “a combined preparation” defines especially a “kit of parts” in the sense that the combination partners as defined above can be used independently or in different combinations i.e., simultaneously, concurrently, separately or sequentially.

### EXAMPLES

### Clinical Trial Design

### Materials and Methods

An Agili-C™ clinical study was conducted assessing performance of the Agili-C™ implant over the current Surgical Standard of Care (SSOC)—microfracture and debridement, for the treatment of joint surface lesions, chondral and osteochondral defects, in the knee joint. The IDE study is a multicenter, 2:1 randomized, open-labeled and controlled trial. 251 subjects (167 in the Agili-C™ arm and 84 in the SSOC arm) were enrolled in 26 sites including sites in the U.S. and outside the U.S. (OUS).

The Agili-C™ was prepared by methods previously described, including methods of preparation, as described in U.S. Pat. No. 11,007,304, 10,806,823 8,808,725 and 8,790,681, and implanted in subjects, as described for example, in U.S. patent application Ser. No. 16/959,447, and in other company publications, each of which is fully incorporated herein by reference, in its entirety.

The primary endpoint for the study was to evaluate the change from baseline to 24 months in average KOOS Overall Score (Pain, Other Symptoms, QOL, ADL and Sports). The KOOS Overall Score ranges from 0 to 100, where higher values represent better outcomes. In addition to the 24-month visit, KOOS Overall Score was measured during intermediate visits at 6 months, 12 months, and 18 months post-baseline.

One goal of the trial was to demonstrate superiority of the Agili-C™ device relative to SSOC by testing the hypothesis:

$$H 0 : θ 1 = θ0 vs . HA : θ1 > θ0 .$$

To test this hypothesis, the posterior probability of superiority: Pr(θ1>θ0|Data) was calculated, with success established if the posterior probability exceeded 0.98 at the final analysis.”

In this formulation, θ1 and θ0 are the mean improvements from baseline to Month 24 in the KOOS Overall Score for subjects randomized to Agili-C™ and SSOC, respectively. The primary and secondary endpoints were to be evaluated in the Full Analysis Set (FAS).

Four confirmatory secondary endpoints were specified to be tested in a hierarchical manner in order to control the type 1 error rate. Each of these secondary endpoints required a Bayesian posterior probability greater than 0.975 for declaring superiority. These endpoints were: change in KOOS Pain score from baseline to Month 24, change in KOOS Quality of Life score from baseline to Month 24, change in KOOS ADL score from baseline to Month 24, and response rate at Month 24 defined as an improvement in KOOS Overall Score≥30.

The safety population consisted of all patients for whom treatment with either Agili-C™ or SSOC was performed, and included N=167 subjects randomized and receiving treatment with Agili-C™ and N=84 subjects randomized and receiving SSOC.

The full analysis set (FAS) consisted of all treated subjects in the Agili-C™ arm and all treated subjects in the SSOC arm, for whom there is a valid overall KOOS score at baseline and at least one valid overall KOOS score post-baseline. A treated subject was defined as any subject who was randomized to any of the groups, and not defined as a subject with major entry violation (as defined below).” Subjects with major entry violations were excluded from FAS.

Major entry violation is defined by having the potential to affect trial results and the decision of whether an entry violation is considered major will be performed by the Medical Director according to the following parameter—enrolment of subject with severe exclusion criteria, such as:
1. Bony defect depth deeper than 8 mm
2. Uncontained lesion—lack of vital bone wall, at least 2 mm thick, completely surrounding the lesion
3. Inability to position the implant 2 mm recessed relative to the articular surface
4. Articular cartilage lesions in the tibia or the patella, ICRS grades IVa or above
5. Systemic cartilage and/or bone disorder”

Three Agili-C™ subjects and one SSOC subject met criteria for exclusion from the FAS. Therefore, the FAS includes N=164 subjects randomized and receiving treatment with Agili-C™ and N=83 subjects randomized and receiving SSOC.

There were no additional exclusions compared to the FAS set as described above due to a major protocol violation. There was one subject in the study, from the Agili-C™ arm, who withdrew consent prior to the Month 12 visit and did not perform the Month 12 visit. Therefore, the PP analysis set includes N=163 subjects randomized and receiving Agili-C™ and N=83 subjects randomized and receiving SSOC. As such, all comparisons were nearly the same for the FAS and the PP analysis set.

Follow-up compliance was excellent. Among N=164 Agili-C™ subjects in the FAS, Month 24 KOOS Overall Scores were available (after BOCF for treatment failures in 160 (97.6%) subjects. Similarly, among N=83 SSOC subjects in the FAS, Month 24 KOOS Overall Scores were available in 79 (95.2%) subjects. Thus, follow-up compliance for the primary effectiveness endpoint was excellent and exceeded 95% in both treatment groups.

251 Subjects were randomized to either receive the Agili-C™ device (167 subjects) or the control arm SSOC (84 subjects) in a 2:1 ratio. Subjects were randomized by site using variable block sizes of 3 and 6. As a consequence of randomization, baseline efficacy self-reported questionnaire scores were nearly identical for both arms: KOOS Overall 41.3 in the Agili-C arm vs. 41.5 in SSOC; Tegner pre-injury 6.1 in Agili-C and 6.0 in SSOC; SF-12 physical score 36.0 in both Agili-C™ and SSOC.

Although most of baseline variables and factors were similar between groups, some clinically significant differences were identified on specific parameters. For example, the percentages of subjects with large lesions, defined as a total lesion area>3 cm², was larger in subjects randomized to Agili-C™ compared to SSOC (58.7% vs 48.8%). Additionally, the percentage of subjects with osteochondral lesions, deeper lesions which involved the subchondral bone and not just the cartilage layer (defined as ICRS grade 4B), was nearly twice as large in subjects randomized to Agili-C™ compared to SSOC (37.7% vs 19.0%). In contrast, the percentage of subjects with Kellgren-Lawrence Grades 2-3, indicating mild/moderate osteoarthritis, was smaller in subjects randomized to Agili-C™ compared to SSOC (45.5% vs 64.3%). The SSOC group was slightly older on average at 46.2 years compared to 42.0 years in the Agili-C™ arm; and had lower mean BMI by about 1.5 kg/m². Since only 3 Agili-C subjects and 1 SSOC was excluded from the FAS, group comparisons at baseline in the FAS were very similar.

### Example 1

### Significantly Low Treatment Failure Rate With Agili-C™ as Compared to SSOC

For the purpose of the study, any secondary invasive treatment in the treated joint, regardless of whether same was related or unrelated to the original treatment, will be considered a treatment failure, in either group. Invasive interventions included: open procedure, mini-open procedure, arthroscopic procedure and intra articular injections, such as: HA, PRP, stem cells and steroids.”

The baseline observation carried forward (BOCF) was used for treatment failures. All observations subsequent to a treatment failure were imputed using BOCF whether or not there was an observed value. One treatment failure occurred in the Agili-C™ group for a subject excluded from the FAS.

In the safety analysis set, 12 of 167 (7.2%) Agili-C™ subjects and 18 of 84 (21.4%) SSOC subjects experienced a treatment failure based on the a priori definition.

The treatment group difference was statistically significant according to an unadjusted chi-square test (p=0.002). Treatment failures in Agili-C™ were due to knee trauma (0 in the SSOC), while 4 of the treatment failures in the SSOC were due to knee replacements and osteotomies (0 in the Agili-C™). Only 5 of 12 (42%) were due increased pain in Agili-C™ compared to 14 of 18 (78%) in SSOC.

A high failure rate was noted in SSOC subjects with mild to moderate OA (27.8% of the subjects), compared to only 5.3% in the Agili-C™ arm.

A similarly high failure rate was noted in SSOC subjects with large lesions (22.0% of the subjects), compared to only 5.1% in the Agili-C™ arm.

It is noteworthy that only 4 of 12 treatment failures in the Agili-C™ group were at least possibly related to the device and/or toolset, with 2 of these only possibly related. In SSOC, all of the treatment failures were at least possibly related to the procedure, although 10 of these were only possibly related.

It is also noteworthy that 15 of 84 (17.9%) SSOC subjects had an intra-articular injection compared with only 3 of 167 (1.8%) Agili-C™ subjects, suggesting a greater failure to adequately reduce pain in SSOC compared to Agili-C™.

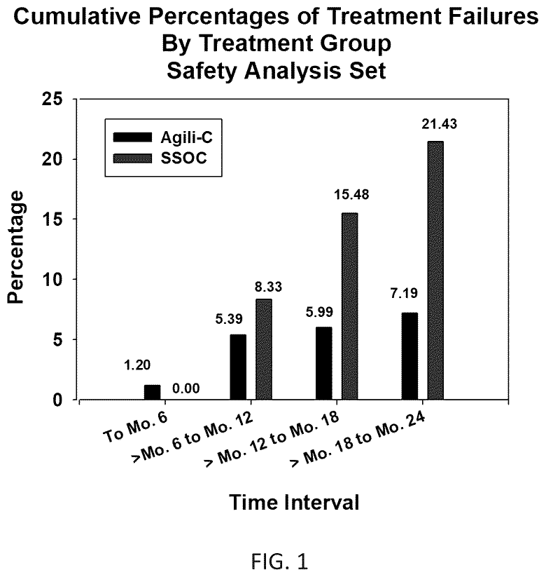

FIG. 1 summarizes the cumulative treatment failure rate.

Notably, while the failures are mostly in the first 6-12 months in the Agili-C™ group, when the subjects are returning to sports activities, in the SSOC group the cumulative failure rate continues to increase over time.

### Example 2

### Dramatic Improvement With Agili-C™ as Compared to SSOC Over Time in KOOS Overall Score

The primary endpoint was the change from baseline to 24 months in the average KOOS Overall Score (Pain, Other Symptoms, QOL, ADL and Sports). The KOOS Overall Score ranges from 0 to 100, where higher values represent better outcomes. In addition to the 24-month visit, KOOS Overall Score was measured during intermediate visits at 6 months, 12 months, and 18 months post-baseline.

The SAS procedure PROC BGLIMM (SAS Institute¹) was used to determine the posterior probability that Agili-C™ was superior to SSOC in terms of mean changes from baseline to Month 24 in KOOS Overall Score. 1 SAS Institute, Cary NC.

The BGLIMM procedure is a sampling-based procedure that provides Bayesian inference for generalized linear mixed models (GLMMs). GLMMs are hierarchical models that combine a generalized linear model with normally distributed random effects. The “Bayesian approach estimates the joint posterior distribution of all parameters in a model, including all fixed-and random-effects parameters. The Monte Carlo method numerically integrates out the random effects and propagates the uncertainties to the marginal posterior of the fixed-effects parameters. PROC BGLIMM uses efficient Markov chain Monte Carlo (MCMC) sampling tools to estimate the posterior marginal distributions and use them for further inference” (SAS® 9.4 Help and Documentation²). 2 SAS/STAT 15.1 User's Guide/Procedures/The BGLIMM Procedure, SAS Institute, Cary NC

Therefore, the statistical model used to determine the posterior probability of superiority was the Bayesian analogue to a Mixed Model for Repeated Measures (MMRM).

The priors on the fixed effects were very diffuse normal distributions. The prior on the covariance matrix was made minimally informative by setting the degrees-of-parameter to a small value (0.5) and by setting the scale parameter to a relatively large value similar in magnitude to the variances observed for KOOS Overall Score changes. The fact that the estimated treatment group contrasts are nearly the same as those obtained using MMRM confirms that the choice of prior had no substantive impact on the results. To provide meaningful within-group estimates from the Bayesian analysis, the values of the baseline score included as a covariate in the model were mean centered based on the same analysis data set that served as input (i.e., FAS or PP as appropriate). This results in a within-group estimate at the mean value of the baseline covariate, which is also consistent with how MMRM constructs within group estimates.

The results from this model when applied to the primary outcome are summarized in Table 4.1.fas. The mean of the posterior distribution for changes from baseline to Month 24 in the KOOS Overall Score for subjects randomized to Agili-C™ is 42.65, with 95% credible interval (or highest posterior density interval) equal to 39.55 to 45.54. For subjects randomized to SSOC, the mean (95% credible interval) of the posterior distribution is 21.39 (17.35, 25.71). It is noteworthy that the upper bound for change in KOOS Overall Score among the SSOC group is smaller than the lower bound for the Agili-C™ group. That is, the 95% credible intervals do not overlap. The mean (95% credible interval) of the posterior distribution for the group difference (Agili-C™ minus SSOC) in mean change from baseline to Month 24 in the KOOS Overall Score is 21.27 (16.17, 26.60).

Based on these results, the posterior probability of superiority was determined to be 1.000. Since 1.000>0.98, the null hypothesis is rejected. Therefore, it is concluded that Agili-C™ is superior to SSOC in terms of improvements from baseline to Month 24 in KOOS Overall Score.

TABLE 4.1.fas
Bayesian Posterior Probability of Month 24 Superiority Agili-C ™ Relative
to SSOC for Primary Effectiveness Endpoint Change from Baseline
to Month 24 in KOOS Overall Score Full Analysis Set

| | | Mean of | SD of | | | Posterior |
|---|---|---|---|---|---|---|
| | | Posterior | Posterior | LB of 95% | UB of 95% | Probability of |
| Parameter | N | Distribution | Distribution | HPD Interval | HPD Interval | Superiority² |
| Agili-C ™ | 5000 | 42.65 | 1.54 | 39.55 | 45.54 | . |
| SSOC | 5000 | 21.39 | 2.14 | 17.35 | 25.71 | . |
| Agili-C ™ - SSOC | 5000 | 21.27 | 2.67 | 16.17 | 26.60 | 1.000 |

Notes:
¹Baseline observation carried forward after treatment failure for 11 Agili-C and 18 SSOCs.
²Posterior probability that the mean improvement is larger for Agili-C compared to SSOC.
Source [btk_MMRM_Bayes.sas]

The Bayesian posterior probability of superiority at Month 24 was determined to be 1.000. The estimated mean improvement was clinically and statistically significantly larger for Agili-C™ compared to SSOC starting at Month 6. The magnitude of the mean improvement increased over time for Agili-C™, but not for SSOC.

At Month 24, the posterior mean for the treatment group improvement from baseline in the Agili-C™ arm was 42.7 compared to only 21.4 for the SSOC arm. The posterior mean of the difference in mean improvements was 21.3 (95% credible interval 16.2 to 26.6). A similar superiority margin was observed among subjects in the FAS with mild-moderate OA (Kellgren-Lawrence Grades of 2 or 3). The superiority margin increased to 27.3 with 95% credible interval of 20.5 to 33.9 for subjects with large lesions (total lesion areas larger than 3 cm2). Results were very similar in the Per Protocol analysis set, which was identical to the FAS analysis set apart from excluding 1 participant randomized to Agili-C™.

In the Safety Analysis Set, a significantly higher rate of treatment failures was observed in the SSOC arm (21.4%) compared to the Agili-C™ arm (7.2%). The estimated treatment group difference was 14.2% (95% CI 4.6% to 23.9%), with an unadjusted chi-square test p=0.001.

A high failure rate was noted in SSOC subjects with mild to moderate OA (27.8% of the subjects), compared to only 5.3% in the Agili-C™ arm.

A similarly high failure rate was noted in SSOC subjects with large lesions (22.0% of the subjects), compared to only 5.1% in the Agili-C™ arm.

It is also noteworthy that 15 of 84 (17.9%) SSOC subjects had an intra-articular injection compared with only 3 of 167 (1.8%) Agili-C™ subjects, suggesting a greater failure to adequately reduce pain in SSOC compared to Agili-C™.

The posterior probability of superiority of Agili-C™ relative to SSOC was also 1.000 for all individual KOOS subscales secondary endpoints (i.e., KOOS Pain, KOOS ADL, KOOS QOL, KOOS Symptoms and KOOS Sports).

Responder rate, which was a-priori defined as improvement of at least 30 points in Overall KOOS at 24 months compared to baseline, was 77.8% in the Agili-C™ arm compared to only 33.6% in the SSOC arm.

Agili-C™'s superiority in effectiveness relative to standard of care confirmed across all subgroups defined by pre-specified covariates.

Remarkably, factors, such as subjects' activity level status of ACL and meniscus, age, type of lesion, size of lesion or number of lesions—which could be expected to negatively impact treatment outcomes due to challenging conditions, did not negatively impact the Agili-C™ superiority over the current surgical standard of care, microfracture and debridement.

Most unexpectedly, BMI, did not negatively impact treatment outcomes for the Agili-C™ whereas currently, subjects suffering from a BMI of over 30 kg/m² do not have any successful treatment option for treating osteoarthritis in this subpopulation group.

Robustness of the findings were confirmed in multiple poolability analyses and in analyses of site-to-site variability.

In addition to the primary and confirmatory secondary endpoints, results with respect to percentage of articular cartilage defect fill according to MRI are particularly notable.

At Month 24, 88.3% of subjects treated with Agili-C™ had at least 75% defect fill compared to only 31.8% among subjects treated with SSOC. Moreover, only 1.3% of the Agili-C™ subjects had less than 50% defect fill at Month 24, compared to 48.5% in the SSOC group.

Only 4 of 164 (2.4%) Agili-C™ and 4 of 83 (4.8%) SSOC were missing Month 24 KOOS Overall Score. A worst-case scenario was defined, and the Bayesian posterior probability of superiority was recomputed. The worst-case scenario replaced missing values at every visit by assigning to Agili-C™ the visit-specific minimum change in KOOS Overall Score observed in the Agili-C™ group and by assigning to SSOC the maximum improvement in KOOS Overall Score observed in the SSOC group. For SSOC, there were some subjects that could not feasibly obtain the maximum observed improvement because their baseline values were too large; and the maximum improvement would imply a value beyond the range of 0 to 100. In these cases, the maximum possible improvement was assigned. Even in this worst-case scenario, the mean (95% credible interval) of the posterior distribution for the group difference in change from baseline to Month 24 in the KOOS Overall Score is 19.01 (13.77, 25.09) and the posterior probability of superiority was equal to 1.000.

Therefore, the superiority conclusion based on the primary effectiveness endpoint is very robust with regard to missing data.

### Example 3

### Continued Improvement Over Time With Agili-C™

The observed KOOS Overall Scores over time were assessed, stratified by treatment group. Group differences are described using 95% confidence intervals (CIs).

There was negligible difference in KOOS Overall Scores at baseline (mean [95% CI] difference=−0.5 [−3.9, 2.9]). Post-baseline, the group differences (95% CI) in mean values increased to 7.3 (2.4, 12.1) at Month 6, 12.1 (6.8, 17.3) at Month 12, 17.8 (12.5, 23.1) at Month 18, and 22.3 (16.9, 27.6) at Month 24. Therefore, all post-treatment values numerically favored the Agili-C™ group at all timepoints, with an increasing advantage for Agili-C™ over time.

Similarly, the group differences (95% CI) in mean change values increased from 8.2 (3.3, 13.0) at Month 6 to 12.5 (7.3, 17.8) at Month 12, 18.3 (13.0, 23.5) at Month 18, and 22.5 (17.0, 28.0) at Month 24.

Due to the non-informative prior distributions used in the Bayesian modeling, it is not surprising that this 95% confidence interval is very similar to the 95% credible interval of 16.2 to 26.6, despite the fact that the 95% confidence interval does not including imputation of missing values.

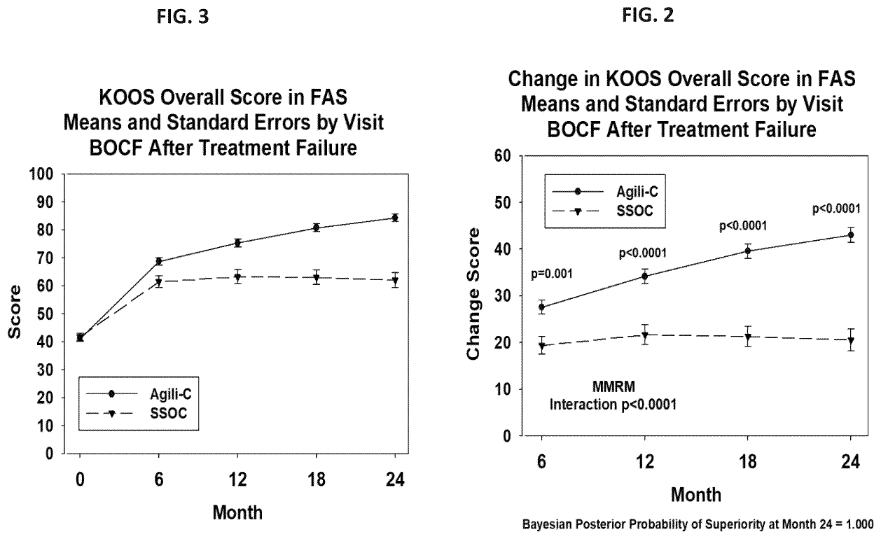

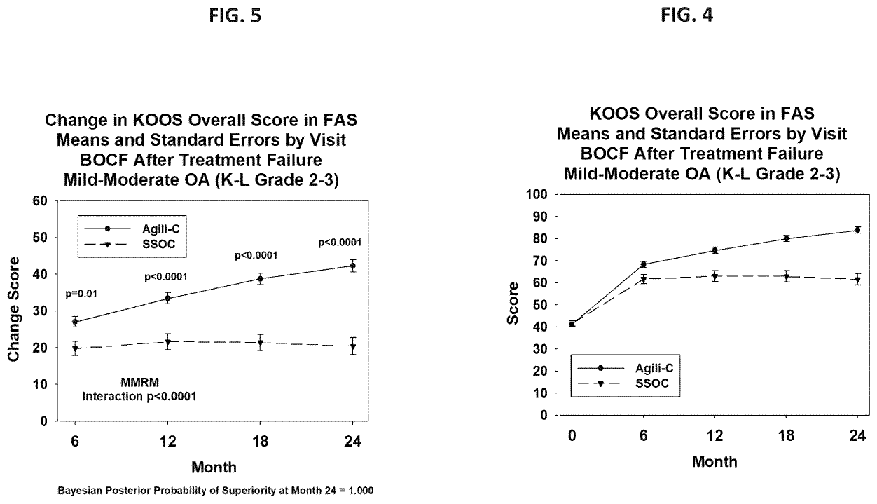

FIGS. 2 and 3 graphically summarize the observed means and mean changes, respectively, including standard errors. FIG. 3 also summarizes the primary Bayesian posterior probability of superiority and the results from the supporting MMRM analyses described above. These graphical analyses further demonstrate that there is continual improvement over time for subjects randomized to Agili-C™. In contrast, after an initial improvement at Month 6, there is no further improvement, on average, for subjects randomized to SSOC.

This observation was formally evaluated using within-group tests for linear trends in mean change in the context of the MMRM and the highly statistically significant linear trend (p<0.0001) in the Agili-C™ group reflects increasing improvements from baseline over time, whereas the non-significant trend (p=0.568) in SSOC reflects no continued improvements from Month 6 through Month 24.

### Example 4

### Analyses of KOOS Overall Score in Subjects With Large Lesions (Total Lesion Area>3 cm2)

Among 164 subjects randomized to Agili-C™, 96 (58.5%) had a total lesion size >3 cm2. Among 83 subjects randomized to SSOC, 41 (49.4%) had a total lesion size>3 cm2.

Within this subgroup, the superiority of Agili-C™ relative to SSOC appeared magnified. The posterior mean improvement in KOOS Overall Score for Agili-C™ was 45.78 compared to 42.65 for the entire FAS. In contrast, the posterior mean improvement for SSOC in this subgroup was only 18.50, compared to 21.39 for the entire FAS. Consequently, the difference (95% credible interval) in the posterior means increased from to 21.27 (16.17, 26.60) in the entire FAS to 27.28 (20.45, 33.93) in the subgroup with total lesion area>3 cm2. The posterior probability of superiority at Month 24 remained at 1.000 despite the smaller sample size.

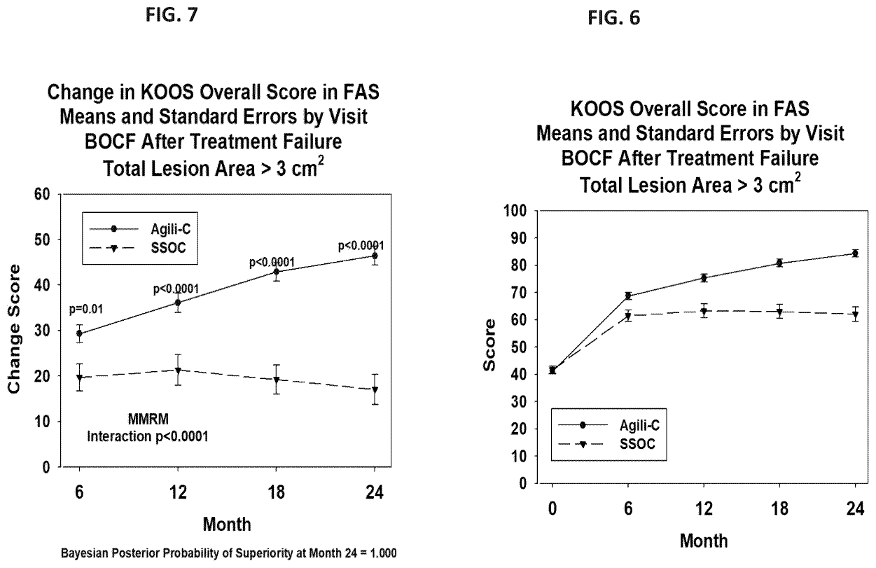

FIG. 6 and FIG. 7 graphically summarize mean values over time and mean changes from baseline in KOOS Overall Score over time in the FAS for subjects with total lesion area>3 cm2.

The Bayesian posterior probability of superiority at Month 24 was determined to be 1.000. The estimated mean improvement was clinically and statistically significantly larger for Agili-C™ compared to SSOC starting at Month 6. The magnitude of the mean improvement increased over time for Agili-C™, but not for SSOC. At Month 24, the posterior mean for the treatment group improvement from baseline was 42.7 in the Agili-C™ arm and 21.4 for the SSOC arm, with difference in mean improvements of 21.3 and 95% credible interval of 16.2 to 26.6.

A similar superiority margin was observed among subjects in the FAS with mild-moderate OA (Kellgren-Lawrence Grades of 2 or 3). The superiority margin increased to 27.3 with 95% credible interval of 20.5 to 33.9 for subjects with large lesions (total lesion areas larger than 3 cm2). Results were very similar in the Per Protocol analysis set, which excluded 1 participant randomized to Agili-C™ but was otherwise identical to the FAS analysis set.

Additionally, in the safety analysis set, a significantly higher rate of treatment failures was observed in the SSOC arm (21.4%, 18/84) compared to the Agili-C™ arm (7.2%. 12/167). The estimated treatment group difference was 14.2% (95% CI 4.6% to 23.9%), with an unadjusted chi-square test p=0.001.

A high failure rate was noted in SSOC subjects with mild to moderate OA (27.8% of the subjects), compared to only 5.3% in the Agili-C™ arm. A similarly high failure rate was noted in SSOC subjects with large lesions (22.0% of the subjects), compared to only 5.1% in the Agili-C™ arm. It is also noteworthy that 15 of 84 (17.9%) SSOC subjects had an intra-articular injection compared with only 3 of 167 (1.8%) Agili-C™ subjects, suggesting a greater failure to adequately reduce pain in SSOC compared to Agili-C™.

### Example 5

### Agili-C™ Improvement Seen Across a Heterogeneity of Treatment Effects

Covariate analyses on the primary endpoint was determined and different age categories were evaluated, wherein Age groups (21-<45, 45-<65, >65) were evaluated, as was Age>50 vs age 50. Other factors assessed included sex, BMI>30 kg/m2 vs <30 kg/m2, Lesion type (chondral or osteochondral), Number of lesions (single or multiple), Level of osteoarthritis (K/L score 0-1 or 2-3), Lesion size (total lesion size<3 cm2 or >3 cm2) Previous ligament reconstruction (with or without) Meniscus status (intact, previous partial meniscectomy, concomitant meniscectomy) and patient Activity status (active or inactive).

MMRM was used to evaluate covariate by treatment group interaction on average changes over time from baseline to Month 24. The MMRM is the same as that specified herein, except that the following additional fixed factors are added:
Covariate
Covariate by group
Covariate by visit
Covariate by visit by group

The focus of these analyses is on the covariate by group interaction. The interpretation of this interaction is a treatment group difference in the mean improvement over time averaging across follow-up visits. The significance of the three-way interaction is also summarized. The three-way interaction reflects covariate differences in the way group differences vary over time.

### MMRM Results

When examining differences based on age, the covariate by group interaction was significant (p=0.014) when comparing subjects aged 21-<45, 45-<65, and >65 years old. In Agili-C™, the MMRM estimated mean improvements from baseline to Month 24 were 42.1, 42.4, and 60.4 across these three age groups. In contrast, in SSOC these mean improvements were 22.2, 21.4, and 0.11, respectively. Consequently, the group differences in these age groups were 19.9, 21.0, and 60.3, respectively.

Furthermore, in Agili-C™, mean improvements to Month 24 in KOOS Overall Scores were 40.7 among males and 46.3 among females. In contrast, these mean values in SSOC were 24.5 among males and 16.3 among females. Consequently, the treatment group difference among females (30.5) was nearly double the group difference observed among males (16.2). The Agili-C™ performed well in both genders, with over 40-point improvement in males and females. Therefore, the interaction appears due to differential effectiveness between genders in SSOC.

Remarkably and also unexpectedly, the covariate by group interaction for BMI had p=0.356, indicating no meaningful difference in the treatment group differences based on obesity.

The covariate by group interaction for osteochondral versus chondral lesions had p=0.1497. The average treatment group difference (95% CI) was 25.1 (14.4, 35.8) for osteochondral lesions and 20.0 (14.0, 26.0) for chondral lesions. Thus, relative treatment effectiveness was similar between lesion types.

Still more unexpectedly, the covariate by group interaction for single versus multiple lesions had p=0.367, indicating no difference in the treatment group differences based on number of lesions.

The covariate by group interaction for K-L Grade had p=0.467, indicating no difference in the treatment group differences based on K-L Grade.

The covariate by group interaction comparing total lesion area>3 cm2 versus <3 cm2 had p=0.101. The treatment group difference was 27.3 for subjects with total lesion area>3 cm2, but only 14.6 for subjects with total lesion are <3 cm2. It is notable that Agili-C™ performed very well in large lesions, which are difficult to treat successfully with available options.

The covariate by group interaction for previous ligament reconstruction had p=0.742, indicating no difference in the treatment group differences based on prior ligament reconstruction.

In the FAS, 35 (21.3%) Agili-C™ subjects had a previous partial meniscectomy. Among these subjects, 12 also had a concomitant procedure on their meniscus, leaving 23 (14.0%) with history only. Similarly, 22 (26.5%) SSOC subjects had a previous partial meniscectomy. Among these subjects, 1 also had a concomitant procedure on their meniscus, leaving 21 (25.3%) with history only. The treatment group differences in mean improvements from baseline to Month 24 were 22.3, 24.2 and 13.6 among subjects with an intact meniscus, history of a previous partial meniscectomy only, and those with a concomitant meniscus procedure. The mean improvements did not statistically significantly differ (interaction p-value=0.457).

Covariate by treatment group interaction based on pre-injury activity status had p=0.115. The group difference in improvements from baseline to Month 24 was 18.9 for subjects that were active pre-injury (defined as a Tegner score>4, N=131 in Agili-C™ and N=61 in SSOC) and was 29.2 for non-active subjects (Tegner score<4, N=33 in Agili-C™ and N=22 in SSOC). This difference was due to improvement in the inactive Agili-C™ subjects, compared to the inactive SSOC subjects that improved significantly less.

In summary, Agili-C™'s superiority in effectiveness relative to standard of care confirmed across all subgroups defined by the pre-specified covariates. Factors, such as subjects' activity level, BMI, status of ACL and meniscus, age, type of lesion, size of lesion or number of lesions—which could be expected to negatively impact treatment outcomes due to challenging conditions, did not negatively impact the Agili-C™ superiority over the current surgical standard of care, microfracture and debridement.

### Example 6

### Agili-C™ Improvement Seen Across Secondary Endpoints

Covariate analyses on the primary endpoint was determined and different age categories were evaluated, wherein Age groups (21-<45, 45-<65, >65) were evaluated, as was Age>50 vs age 50. Other factors assessed included sex, BMI>30 kg/m2 vs <30 kg/m2, Lesion type (chondral or osteochondral), Number of lesions (single or multiple), Level of osteoarthritis (K/L score 0-1 or 2-3), Lesion size (total lesion size<3 cm2 or >3 cm2) Previous ligament reconstruction (with or without) Meniscus status (intact, previous partial meniscectomy, concomitant meniscectomy) and patient Activity status (active or inactive).

The following four confirmatory secondary endpoints to be tested in a hierarchical manner in order to control the type 1 error rate. Each of these secondary endpoints requires a Bayesian posterior probability greater than 0.975 for declaring superiority.

The four pre-specified confirmatory secondary endpoints are:
Change in KOOS Pain score from baseline to Month 24
Change in KOOS Quality of Life score from baseline to Month 24
Change in KOOS ADL score from baseline to Month 24
Response rate at Month 24 defined as an improvement in KOOS Overall Score>30

### Change in KOOS Pain Score From Baseline to Month 24

Results for the first confirmatory endpoint, change in KOOS Pain score from baseline to Month 24 indicate that the mean posterior (95% credible interval) for the group difference in changes was 20.33 (15.37, 25.05). The posterior probability of superiority was 1.000, which is larger than the pre-specified 0.975.

### Change in KOOS Quality of Life Score From Baseline to Month 24

Results for the second confirmatory endpoint, change in KOOS Quality of Life score from baseline to Month 24 indicate that the mean posterior (95% credible interval) for the group difference in changes was 23.79 (17.01, 30.44). The posterior probability of superiority was 1.000, which is larger than the pre-specified 0.975.

### Change in KOOS ADL Score From Baseline to Month 24

Results for the third confirmatory endpoint, change in KOOS ADL score from baseline to Month 24 indicate that the mean posterior (95% credible interval) for the group difference in changes was 19.25 (14.60, 23.84). The posterior probability of superiority was 1.000, which is larger than the pre-specified 0.975.

### Response Rate at Month 24

Results for the fourth confirmatory endpoint, improvement in KOOS Overall Score>30 from baseline to Month 24 were assessed. Given the binary confirmatory endpoint, analyses were performed utilizing a Bayesian Multiple Imputation (MI), with results presented as the average mean posterior and average 95% non-parametric credible interval across imputations. For participants with missing Month 24 outcomes, the Bayesian MI algorithm imputes the expected outcome at Month 24 using a treatment-specific beta-binomial distribution to model the transition probability from the outcome at the last observed timepoint (month j) to outcome at Month 24. Using Jeffrey's prior, β(0.5,0.5), the probability of success for a subject in treatment group t, with last follow-up month j and last responder status r (e.g., πt,j,r) can be described using the beta distribution β(0.5+St,j,r, 0.5+Ft,j,r), where St.j,r represents the number of observed Month 24 successes and Ft,j,r the number of observed Month 24 failures in treatment group t that had responder status r at follow-up month j. A randomly chosen πt,j,r from this beta distribution is assigned to each subject with missing Month 24 outcome, and their final responder status is imputed from a binomial distribution Bin(n, p), with n=1 and p=πt,j,r, to obtain a complete dataset. The total number of observed and imputed successes (St) and failures (Ft) in each treatment group t is used to generate the posterior beta distributions, β(0.5+St, 0.5+Ft). Using repeated (n=5,000) random draws from these treatment-specific distributions, the posterior distribution of differences in treatment responder rate is calculated. This process is repeated 20 times to complete the multiple imputation. Results are then averaged to determine the final posterior probability of superiority and associated summaries of the posterior distributions.

Using this approach, the mean posterior (95% credible interval) for the group difference in response rate was 0.443 (0.320, 0.557) (corresponding to a 77.8% response rate for Agili-C™ compared to only 33.6% for SSOC). The posterior probability of superiority was 1.000, which is larger than the pre-specified 0.975.

As described above, the posterior probabilities of superiority for all 4 confirmatory secondary endpoints were 1.000. Since all of these values were greater than 0.975, it can be concluded that Agili-C™ is superior to SSOC in terms of improvements from baseline to Month 24 in KOOS Pain scores, KOOS Quality of Life scores, KOOS ADL scores, and the likelihood of achieving at least a 30-point increase in the KOOS Overall Score.

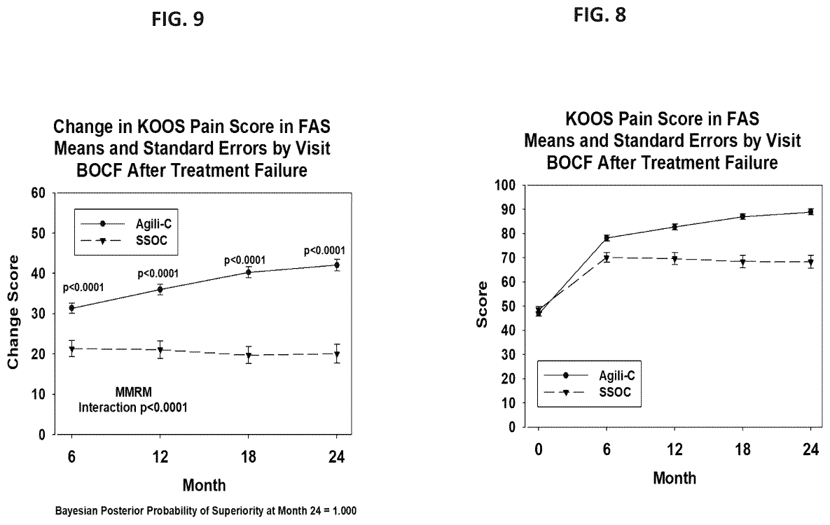

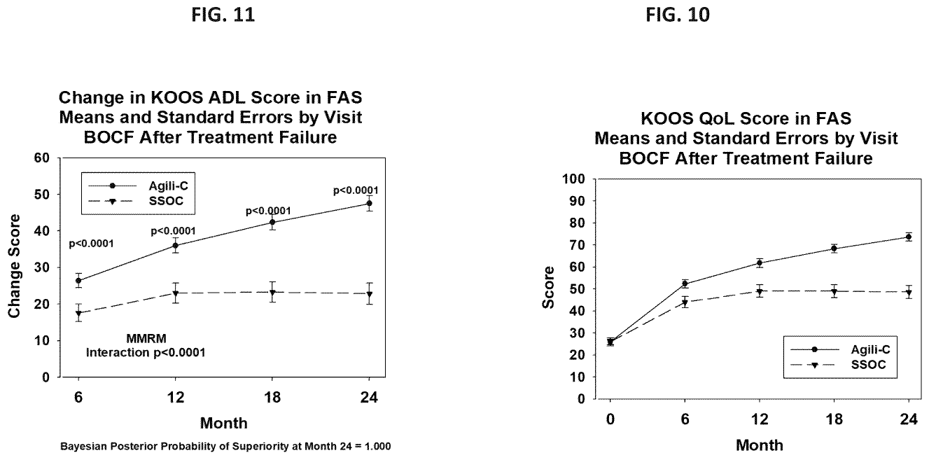

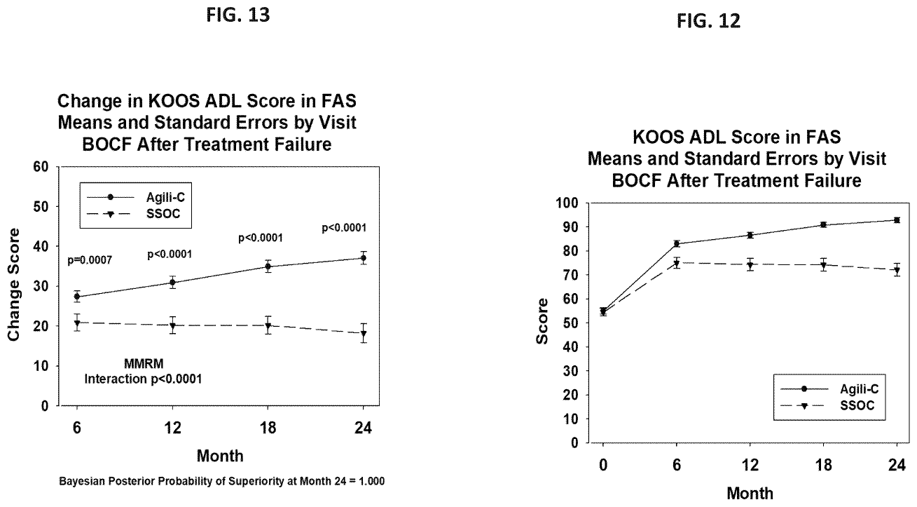

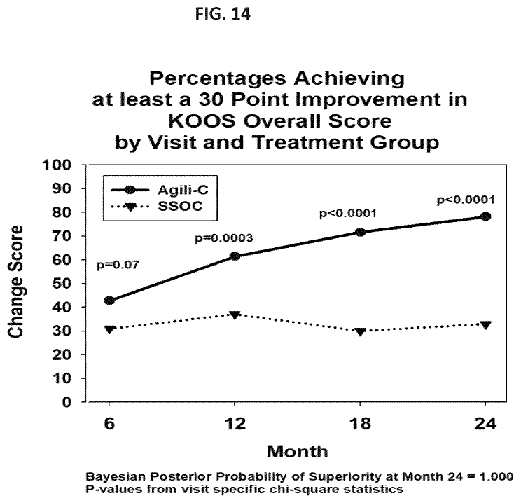

FIGS. 8-14 provide graphical summaries of the values and changes from baseline over time for confirmatory secondary endpoints of KOOS Pain, KOOS Quality of Life, and KOOS ADL scores, as well as the percentages of subjects achieving at least a 30-point improvement in KOOS Overall Score.

It is noted that for each confirmatory endpoint there is statistically significant evidence (p<0.0001) of increasing improvements over time among the Agili-C™. For SSOC, only KOOS Quality of Life shows evidence of increased improvements over time (p=0.031), with results appearing to plateau after 12 months (rather than after 6 months as seen in other outcomes). For each secondary confirmatory endpoint, there were larger differences in improvements over time in Agili-C™ compared to SSOC (p<0.0001).

Furthermore, for each confirmatory endpoint there is statistically significant evidence (p<0.0001) of increasing improvements over time among the Agili-C™, but not in SSOC, and larger differences in improvements over time compared to SSOC.

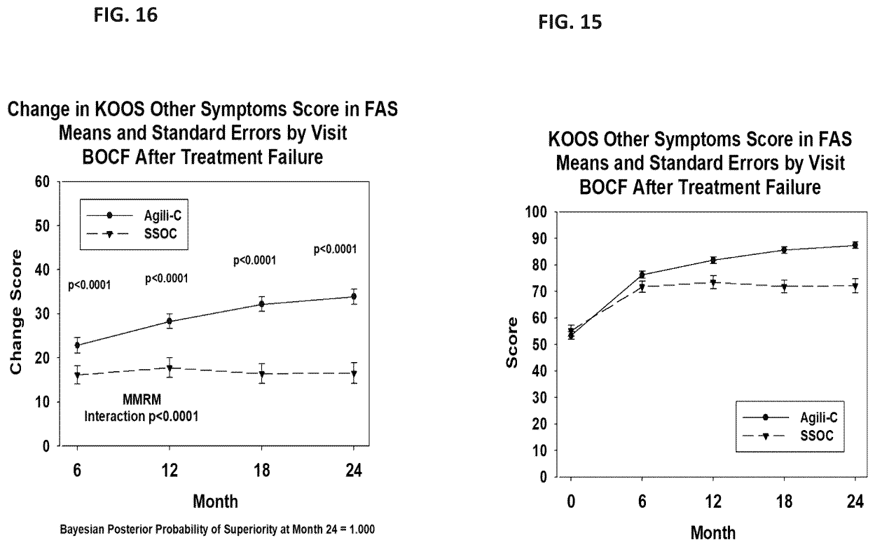

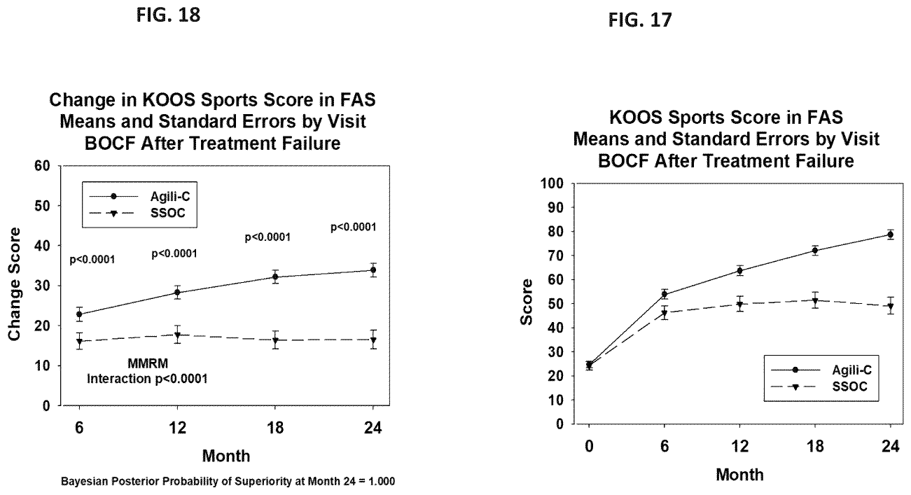

Graphical analyses of KOOS Other Symptoms Score and KOOS Sports Score values and changes over time are provided in FIGS. 15, 16, 17, and 18.

Results further illustrate the conclusions described in the Bayesian and MMRM analyses.

Percentages of articular cartilage defect fill were evaluated by MRI and categorized as 0-24%, 25-49%, 50-74%, 75-99%, and 100%. The percentages were obtained by averaging scores across readers and then assigning the mean score to the corresponding category. Analyses were performed based on MRI at Month 12 and at Month 24.

To preserve the ordinal nature of the categories, group comparisons were performed using a Wilcoxon rank sum test at each timepoint.

The distributions of MRI defect fill were very different and very highly statistically significant (<0.0001) between Agili-C™ and SSOC. Results were similar at Months 12 and 24.

Specifically, subjects treated with Agili-C™ were more likely to have a higher percentages of articular cartilage defect fill according on MRI than subjects receiving SSOC at both 12 and 24 months.

For example, at Month 24 a total of 88.3% of subjects treated with Agili-C™ had at least 75% defect fill compared to only 31.8% of subjects treated with SSOC. Moreover, only 1.3% of the Agili-C™ subjects had less than 50% defect fill at Month 24, compared to 48.5% in the SSOC group.

Implantation of the Agili-C™ implant is performed in an arthrotomy procedure which involve knee opening, osteochondral drilling to create a designated implantation site and implants placement. In contrast SSOC procedures are conducted through mini-invasive arthroscopy procedures. Nevertheless, when comparing the safety results the group differences are all negative reflecting a favorable safety profile for the Agili-C™ implant and its related procedure. In several cases, the upper bound of the 95% confidence intervals are less than zero, suggesting a superior safety profile.

In terms of AEs 58.7% in the Agili-C arm experienced at least one adverse event, compared to 77.4% of the subjects in the SSOC group; the most common AE was increased transient chronic knee pain. This AE was presented in 15.0% of the subjects in the Agili-C™ arm compared 39.3% of the SSOC subjects.

In term of AEs 58.7% in the Agili-C arm experienced at least one adverse event, compared to 77.4% of the subjects in the SSOC group; the most common AE was increased transient chronic knee pain. This AE was presented in 15.0% of the subjects in the Agili-C™ arm compared 39.3% of the SSOC subjects.

At least one Severe AEs was presented in 9.6% of the Agili-C™ subjects compared to 20.2% in the SSOC, and at least one Serious AEs were presented in 15.6% of the Agili-C™ subjects compared to 20.2% in the SSOC.

These results describe the results of primary and secondary efficacy analyses for Protocol CLN0021 (CartiHeal Ltd.), evaluating the superiority of Agili-C™ compared to surgical standard of care (SSOC) for the treatment of joint surface lesions of the knee.

All data strongly support the superiority of Agili-C™ over SSOC.

Follow-up compliance was excellent. Among N=164 Agili-C™ subjects in the FAS, Month 24 KOOS Overall Scores were available (after BOCF for treatment failures) in 160 (97.6%) subjects. Similarly, among N-83 SSOC subjects in the FAS, Month 24 KOOS Overall Scores were available in 79 (95.2%) subjects. Thus, follow-up compliance for the primary effectiveness endpoint was excellent, exceeding 95% in both treatment groups.

The Bayesian posterior probability of superiority in term of the primary effectiveness endpoint, change from baseline to Month 24 in the KOOS Overall Score, was determined to be 1.000.

The estimated mean improvement was clinically and statistically significantly larger for Agili-C™ compared to SSOC starting at Month 6. The magnitude of the mean improvement increased over time for Agili-C™, but not for SSOC. At Month 24, the posterior mean for the treatment group improvement from baseline in the Agili-C™ arm was 42.7 compared to only 21.4 for the SSOC arm. The posterior mean of the difference in mean improvements was 21.3 (95% credible interval 16.2 to 26.6). A similar superiority margin was observed among subjects in the FAS with mild-moderate OA (Kellgren-Lawrence Grades of 2 or 3). The superiority margin increased to 27.3 with 95% credible interval of 20.5 to 33.9 for subjects with large lesions (total lesion areas larger than 3 cm2). Results were very similar in the Per Protocol analysis set, which was identical to the FAS analysis set apart from excluding 1 participant randomized to Agili-C™.

In the Safety Analysis Set, a significantly higher rate of treatment failures was observed in the SSOC arm (21.4%) compared to the Agili-C™ arm (7.2%). The estimated treatment group difference was 14.2% (95% CI 4.6% to 23.9%), with an unadjusted chi-square test p=0.001.

A high failure rate was noted in SSOC subjects with mild to moderate OA (27.8% of the subjects), compared to only 5.3% in the Agili-C™ arm. A similarly high failure rate was noted in SSOC subjects with large lesions (22.0% of the subjects), compared to only 5.1% in the Agili-C™ arm. It is also noteworthy that 15 of 84 (17.9%) SSOC subjects had an intra-articular injection compared with only 3 of 167 (1.8%) Agili-C™ subjects, suggesting a greater failure to adequately reduce pain in SSOC compared to Agili-C™.

The posterior probability of superiority of Agili-C™ relative to SSOC was also 1.000 for all individual KOOS subscales secondary endpoints (i.e, KOOS Pain, KOOS ADL, KOOS QOL, KOOS Symptoms and KOOS Sports).

Responder rate, which was a-priori defined as improvement of at least 30 points in Overall KOOS at 24 month compared to baseline was 77.8% in the Agili-C™ arm compared to only 33.6% in the SSOC arm.

Agili-C™'s superiority in effectiveness relative to standard of care was confirmed across all subgroups defined by pre-specified covariates. Factors, such as subjects' activity level, BMI, status of ACL and meniscus, age, type of lesion, size of lesion or number of lesions—which could be expected to negatively impact treatment outcomes due to challenging conditions, did not negatively impact the Agili-C™ superiority over the current surgical standard of care, microfracture and debridement.

Robustness of the findings were confirmed in multiple poolability analyses and in analyses of site-to-site variability.

In addition to the primary and confirmatory secondary endpoints, results with respect to percentage of articular cartilage defect fill according to MRI are particularly notable. At Month 24, 88.3% of subjects treated with Agili-C™ had at least 75% defect fill compared to only 31.8% among subjects treated with SSOC. Moreover, only 1.3% of the Agili-C™ subjects had less than 50% defect fill at Month 24, compared to 48.5% in the SSOC group.

### Example 7

### Agili-C™ Improvement Representative Patient Results

In the study described hereinabove, a 30 year old female subject with a BMI of 23.6 presented with a single MFC Central lesion (6 cm², 2×7.5 mm), presenting with synovitis and having a K/L scopre of 1 was implanted with two Agili-C implants, 7.5 mm each.

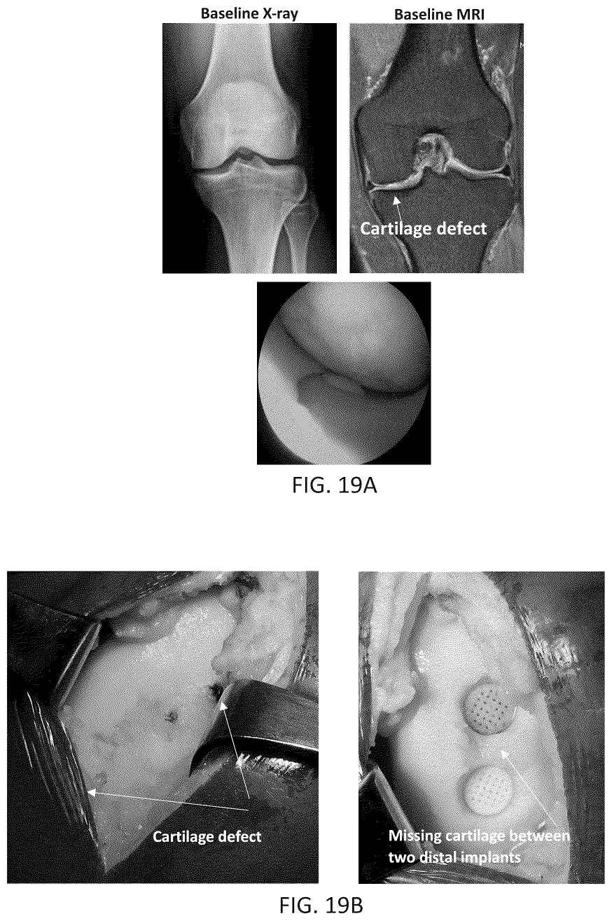

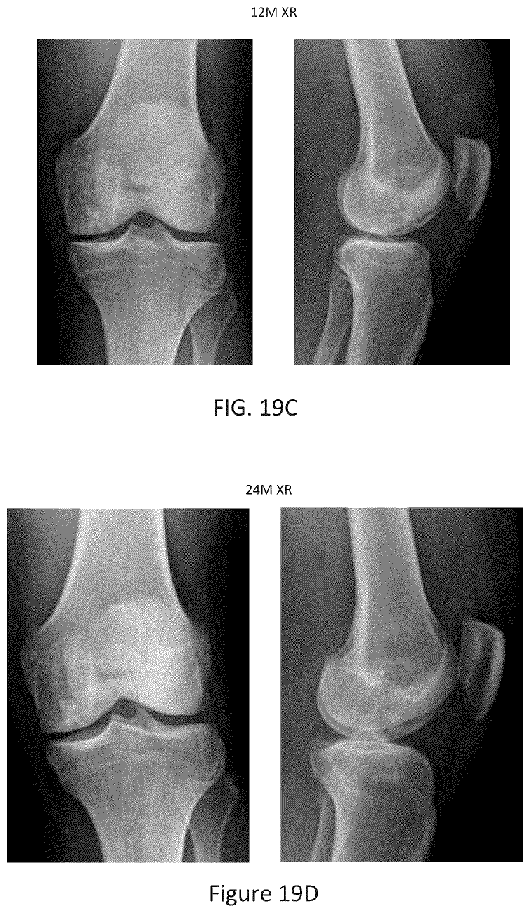

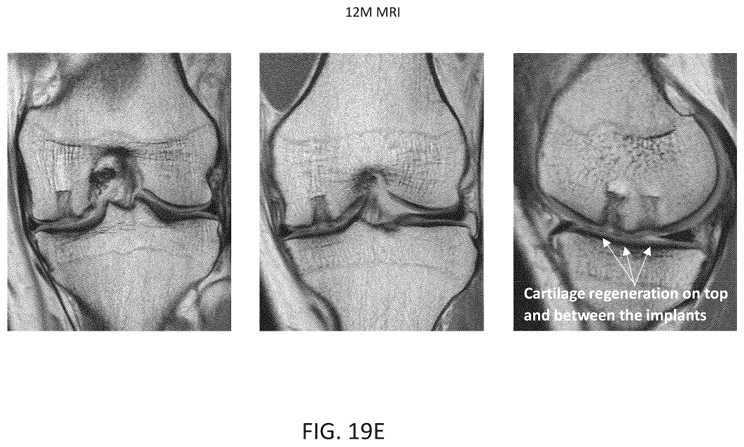

FIG. 19A shows baseline status by X-ray and MRI, with the arrow indicating the region of cartilage defect. FIG. 19B highlights further the extent of the cartilage defect and implantation of two implants, where the implants were placed within the defect site, but in an area bordering on/adjacent to healthy tissue and the significantly damaged region missing cartilage located between the two implants.

FIGS. 19C and 19D show X-ray results 12 and 24 months after the procedure, where full healing is evident, the results being as readily evident from the 12 month MRI results shown in

FIG. 19E. Full cartilage regeneration along the defect site, even in the region between the two implants is shown.

Table A plots the comparative scoring values at baseline as compared to 6, 12, 18 and 24 months post implantation.

TABLE A

| | Baseline | 6 M | 12 M | 18 M | 24 M |
|---|---|---|---|---|---|
| IKDC | 45.98 | 67.82 | 97.70 | 90.80 | 100 |
| KOOS Pain | 61.11 | 94.44 | 100 | 100 | 100 |
| KOOS QOL | 31.25 | 93.75 | 100 | 100 | 100 |
| KOOS Symptoms | 71.43 | 82.14 | 96.43 | 100 | 100 |
| KOOS ADL | 72.06 | 98.53 | 100 | 100 | 100 |
| KOOS Sport | 10 | 75 | 100 | 100 | 100 |
| KOOS Overall | 49.17 | 88.77 | 99.29 | 100 | 100 |

Another representative positive study subject result from the study described hereinabove, relates to a 35 year old male subject with a BMI of 24.7 presented with an osteochondral MFC Central lesion (4 cm²), who had undergone concomitant patella-ostophyte removal, and a crystal was found in the area of the lesion from a previous implantation procedure 15 years prior to his presentation. This subject presented with a K/L Score of 1 and was implanted with two Agili-C implants (12.5 mm and 15 mm).

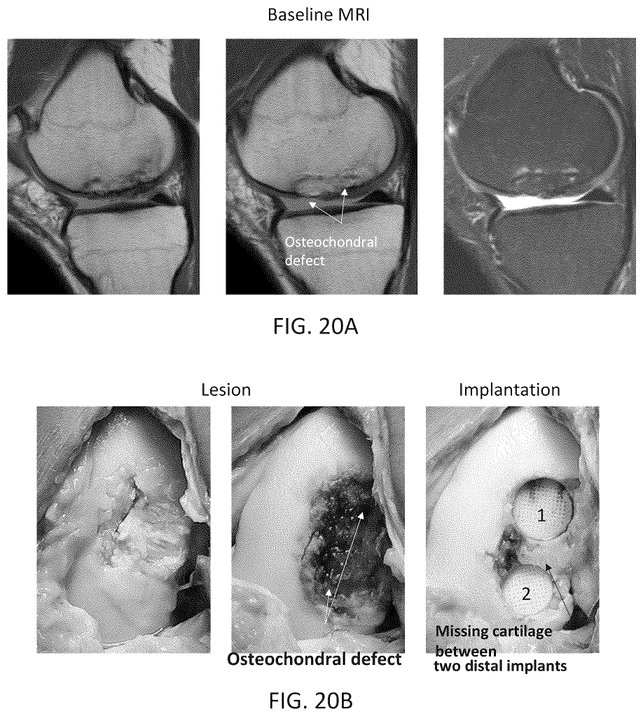

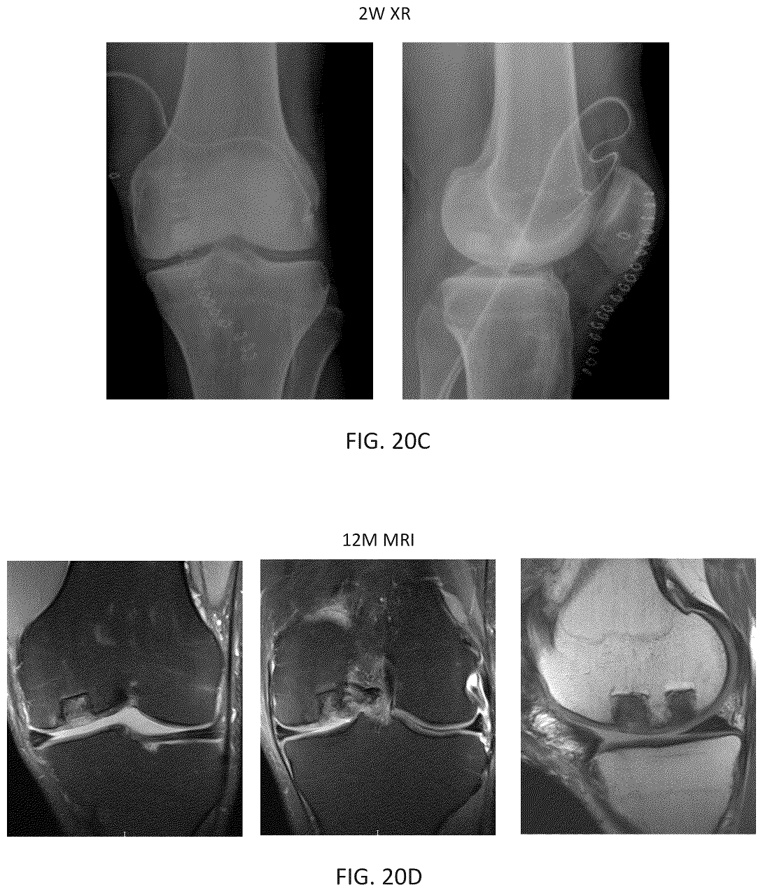

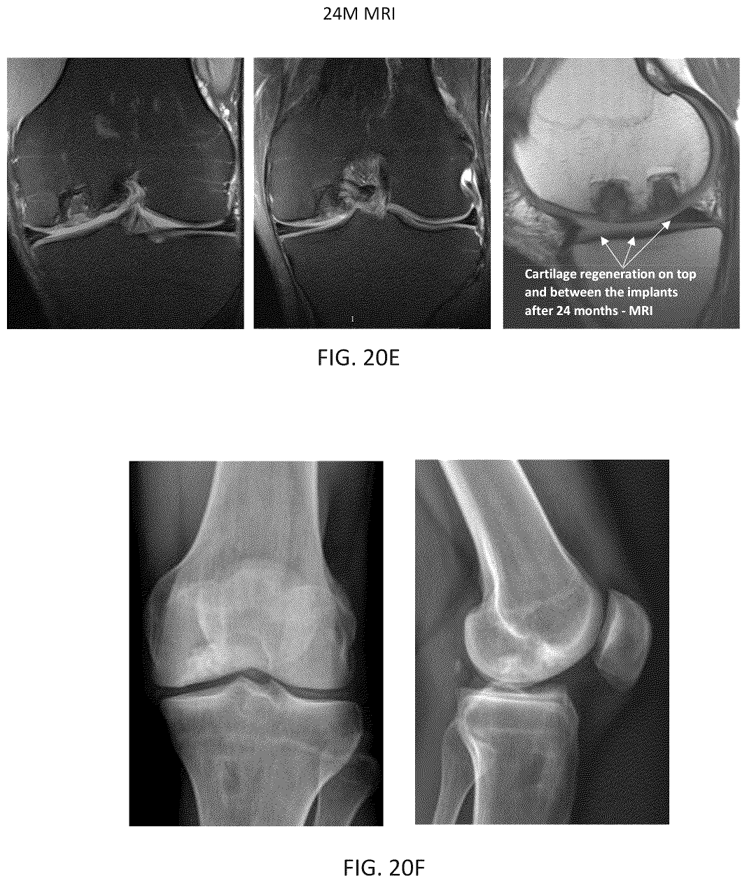

FIG. 20A shows baseline status by MRI, with the arrow indicating the region of osteochondral defect. FIG. 20B highlights further the extent of the cartilage defect and implantation of two implants, where the implants were placed within the defect site, but in an area bordering on/adjacent to healthy tissue and the significantly damaged region missing cartilage located between the two implants.

FIG. 20C shows X-ray results 2 weeks after the procedure. FIGS. 20D and 20E show MRI results 12 and 24 months post-implantation. By 24 months cartilage regeneration is readily evident as shown by the arrows on the figure. FIG. 20F shows X-ray results from 36 months post implantation and full healing is evident. Full cartilage regeneration along the defect site, even in the region between the two implants is shown.

Table B plots the comparative scoring values at baseline as compared to 6, 12, 18 and 24 months post implantation.).

described hereinabove, relates to a 50 year old male subject with a BMI of 29.7 presented with two osteochondral LFC lesions (3.8 cm² total in size). This subject presented with a K/L Score of 3 and was implanted with two Agili-C implants (7.5 mm and 10 mm).

TABLE B

| | Baseline | 6 M | 12 M | 18 M | 24 M | 36 M |
|---|---|---|---|---|---|---|
| IKDC | 62.07 | 81.61 | 95.40 | 97.70 | 95.40 | 97.70 |
| KOOS Pain | 61.11 | 91.67 | 100 | 100 | 100 | 100 |
| KOOS QOL | 43.75 | 62.5 | 93.75 | 93.75 | 100 | 87.5 |
| KOOS | 78.57 | 89.29 | 96.43 | 96.43 | 100 | 96.43 |
| Symptoms | | | | | | |
| KOOS ADL | 86.76 | 98.53 | 100 | 100 | 100 | 100 |
| KOOS Sport | 60 | 100 | 100 | 100 | 100 | 100 |
| KOOS | 66.04 | 88.40 | 98.04 | 98.04 | 100 | 96.79 |
| Overall | | | | | | |

Another representative positive study subject result from the study

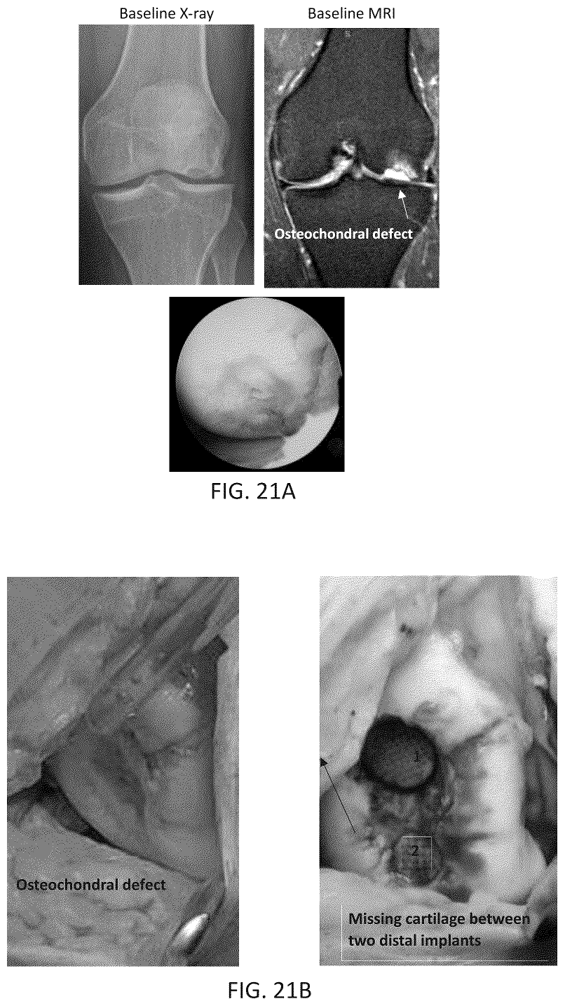

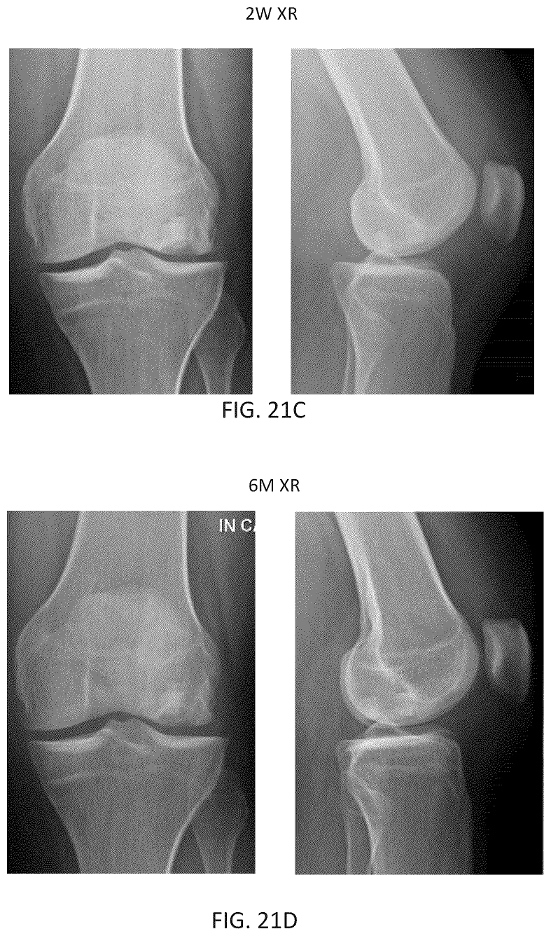

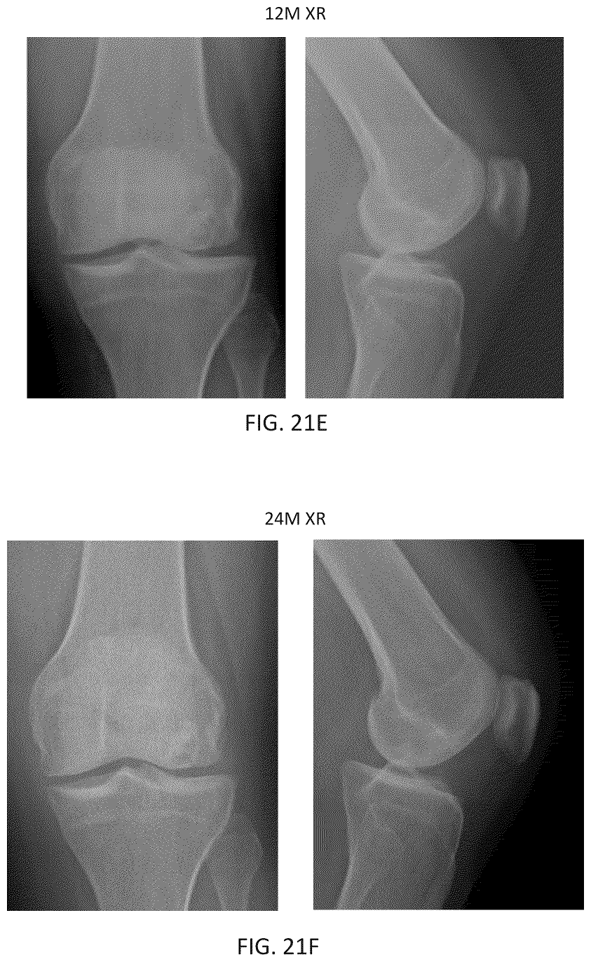

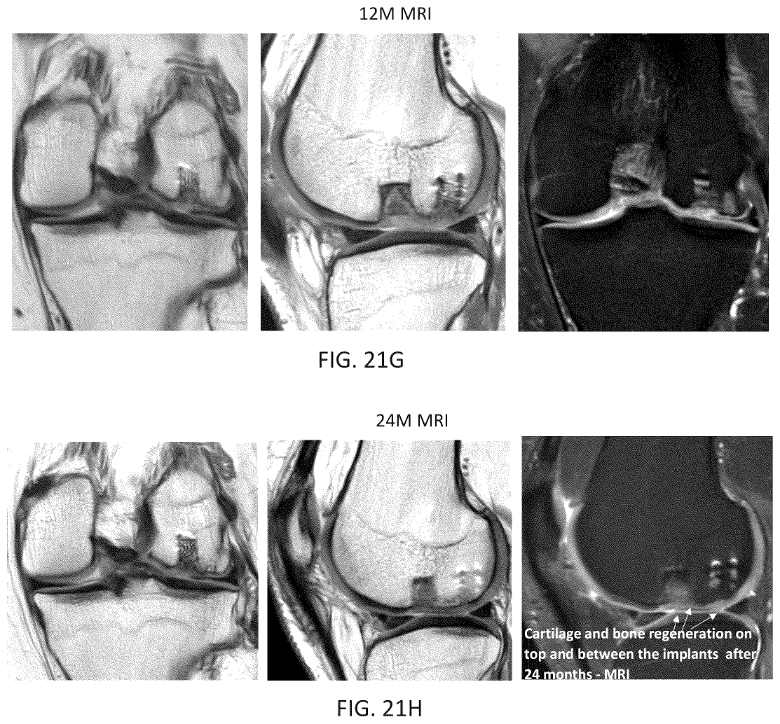

FIG. 21A shows baseline status by X-ray and MRI, with the arrow indicating the region of osteochondral defect. FIG. 21B highlights further the extent of the cartilage defect and implantation of two implants, where the implants were placed within the defect site, but in an area bordering on/adjacent to healthy tissue and the significantly damaged region missing cartilage located between the two implants.

FIGS. 21C-21F show X-ray results 2 weeks, 6 months, 12 and 24 months, respectively, after the procedure. FIGS. 21G and 21H show MRI results 12 and 24 months post-implantation. By 24 months cartilage and bone regeneration is readily evident as shown by the arrows on the figure. FIG. 20F shows X-ray results from 36 months post implantation and full healing is evident. Full cartilage regeneration along the defect site, even in the region between the two implants is shown.

Uniquely, looking at the left panels of 21G and 21H, bone regeneration followed the pattern of the architecture of the coral implant.

Table C plots the comparative scoring values at baseline as compared to 6, 12, 18 and 24 months post implantation.).

TABLE C

| | Baseline | 6 M | 12 M | 18 M | 24 M |
|---|---|---|---|---|---|
| IKDC | 44.83 | 82.76 | 86.21 | 79.31 | 90.80 |
| KOOS Pain | 52.78 | 94.44 | 94.44 | 86.11 | 100 |
| KOOS QOL | 31.25 | 81.25 | 93.75 | 62.5 | 100 |
| KOOS Symptoms | 64.29 | 92.86 | 100 | 85.71 | 100 |
| KOOS ADL | 64.71 | 98.53 | 98.53 | 95.59 | 100 |
| KOOS Sport | 40 | 80 | 100 | 75 | 100 |
| KOOS Overall | 50.61 | 89.42 | 97.34 | 80.98 | 100 |

Taken together these representative patient descriptions demonstrate the unexpected results as described herein and provide support for the methods and uses of the invention as described and claimed.

It will be understood by those skilled in the art that various changes in form and details may be made therein without departing from the spirit and scope of the invention as set forth in the appended claims. Those skilled in the art will recognize, or be able to ascertain, using no more than routine experimentation, many equivalents to the specific embodiments of the invention described herein. Such equivalents are intended to be encompassed in the scope of the claims.

In one embodiment of this invention, “about” refers to a quality wherein the means to satisfy a specific need is met, e.g., the size may be largely but not wholly that which is specified but it meets the specific need of cartilage repair at a site of cartilage repair. In one embodiment, “about” refers to being closely or approximate to, but not exactly. A small margin of error is present. This margin of error would not exceed plus or minus the same integer value. For instance, about 0.1 micrometers would mean no lower than 0 but no higher than 0.2. In some embodiments, the term “about” with regard to a reference value encompasses a deviation from the amount by no more than 5%, no more than 10% or no more than 20% either above or below the indicated value.

In the claims articles such as “a”, “an” and “the” mean one or more than one unless indicated to the contrary or otherwise evident from the context. Claims or descriptions that include “or” or “and/or” between members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides, in various embodiments, all variations, combinations, and permutations in which one or more limitations, elements, clauses, descriptive terms, etc., from one or more of the listed claims is introduced into another claim dependent on the same base claim unless otherwise indicated or unless it would be evident to one of ordinary skill in the art that a contradiction or inconsistency would arise. Where elements are presented as lists, e.g. in Markush group format or the like, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in haec verba herein. Certain claims are presented in dependent form for the sake of convenience, but Applicant reserves the right to rewrite any dependent claim in independent format to include the elements or limitations of the independent claim and any other claim(s) on which such claim depends, and such rewritten claim is to be considered equivalent in all respects to the dependent claim in whatever form it is in (either amended or unamended) prior to being rewritten in independent format.

What is claimed is:

1. A method of treating or repairing a large osteochondral, bone or cartilage lesion site in a subject in need thereof, said method comprising:
implanting two or more solid substrates comprising a coral or coral derivative within a region in said lesion site in said subject, wherein:
said defect site has a length or width or depth that is at least about 1.5 times greater than a length, width or depth of said two or more solid substrates being implanted therein;
said two or more solid substrates consisting essentially of two phases wherein:
a first phase of said two phases comprises solid coral and said first phase further comprises a series of hollows along a longitudinal axis in said first phase;
a second phase of said two phases comprises a solid coral;
said two or more solid substrates being characterized by a specific fluid uptake capacity value of at least 75% or characterized by having a contact angle value of less than 60 degrees, when in contact with a fluid; and
said two or more solid substrates are further characterized by each having at least one substantially flat cross section at a terminus of said solid substrate and tapered sides, which tapered sides are at an angle of from 0.75 to about 4 degrees from a longitudinal axis along said solid substrate.

2. The method according to claim 1, wherein said lesion site is larger than 3 cm2.

3. The method according to claim 2, wherein said two or more solid substrates are placed near opposite boundaries of said lesion area larger than 3 cm2.

4. The method according to claim 1, wherein said two or more solid substrates are placed at least at about a 3 mm distance from each other along a Cartesian axis.

5. The method according to claim 1, wherein said two or more solid substrates are placed at spaced intervals of at least about 3 mm in distance to span said lesion area.

6. The method according to claim 1, wherein said two or more solid substrates do not fill more than 90% of the total area of said lesion site.

7. The method according to claim 1, wherein said two or more solid substrates are positioned at or near a diseased or affected tissue site and wherein said two or more solid substrates are also proximally located to healthy cartilage and/or bone tissue.

8. The method according to claim 7, wherein proximity of said two or more solid substrates to healthy cartilage and/or bone tissue promotes migration of healthy cells and/or participation of therapeutic factors from within the healthy tissue in said treating or repairing a large osteochondral, bone or cartilage lesion site in a subject in need thereof.

9. The method according to claim 1, wherein said hollows have a diameter ranging from about 125 to 650 mm.

10. The method according to claim 1, wherein said first phase has a height of between 1-7 mm.

11. The method according to claim 1, wherein said method promotes cartilage regeneration, healing or a combination thereof in regions not in immediate contact with said region in which said solid substrates are placed within said lesion, thereby being a method of treating or repairing a large osteochondral, bone or cartilage lesion site in a subject in need thereof.

12. The method according to claim 1, wherein said method promotes cartilage regeneration, healing or a combination thereof in regions not proximal to said region in which said solid substrates are placed within said lesion, thereby being a method of treating or repairing a large osteochondral, bone or cartilage lesion site in a subject in need thereof.

13. The method according to claim 1, wherein said method promotes significant cartilage regeneration, healing or a combination thereof in said subject within six months as compared to microfracture and debridement procedures.

14. The method according to claim 1, wherein said method promotes and/or sustains significant cartilage regeneration, healing or a combination thereof in said subject twelve months or more, as compared to microfracture and debridement procedures.

15. The method according to claim 1, wherein said method promotes bone regeneration, healing or a combination thereof in regions not proximal to said region in which said solid substrates are placed within said lesion.

16. The method according to claim 1, wherein said two or more solid substrates are positioned anywhere by or near the periphery of said lesion site and said two or more solid substrates do not substantially fill said lesion site.

17. The method according to claim 1, wherein said two or more solid substrates are positioned at any relative position with respect to each other within said lesion site.

18. The method according to claim 1, wherein said subject suffers from a full or partial thickness articular cartilage defect; osteochondral defect; osteoarthritis; osteochondritis dissecans; a joint defect; or a defect resulting from trauma, sports, or repetitive stress.

19. The method according to claim 1, wherein said two or more solid substrates are placed such that said at least a second porous phase is implanted within or proximally to cartilage tissue and said at least a first porous phase is implanted within or proximally to bone tissue.

20. The method according to claim 1, further comprising the step of establishing a specific fluid uptake capacity value of said solid substrate, comprising contacting said solid substrate with a fluid for from 0.1-15 minutes, allowing for spontaneous fluid uptake of said fluid within said solid substrate to arrive at said spontaneous fluid uptake value.

21. The method according to claim 1, further comprising the step of establishing a specific fluid uptake capacity value of said solid substrate comprising contacting said solid substrate with a fluid and applying negative pressure to said solid substrate to promote maximal uptake of said fluid within said solid substrate to arrive at a total fluid uptake value.

22. The method according to claim 1, wherein said subject suffers from a full or partial thickness articular cartilage defect; osteochondral defect; osteoarthritis, a joint defect or a defect resulting from trauma, sports, or repetitive stress.

23. The method according to claim 1, wherein said subject suffers from a bone fracture, bone defect, bone edema, osteoporosis, or a defect resulting from trauma, sports, or repetitive stress.

24. The method according to claim 1, wherein said method serves to resurface an affected joint in a subject.

25. A method of treating osteochondral, bone or cartilage disease in a subject from a population at greater risk for same, said method comprising:
identifying a subject from a population at greater risk for osteochondral, bone or cartilage disease and selecting said subject in need of treatment;
implanting at least one solid substrate comprising a coral or coral derivative within at least one affected region in bone, cartilage or osteochondral tissue in said subject, wherein:
said at least one solid substrate being characterized by a specific fluid uptake capacity value of at least 75% or characterized by having a contact angle value of less than 60 degrees, when in contact with a fluid; and
said at least one solid substrate is further characterized by each having at least one substantially flat cross section at a terminus of said solid substrate and tapered sides, which tapered sides are at an angle of from 0.75 to about 4 degrees from a longitudinal axis along said solid substrate.

26. The method of claim 25, wherein said population at greater risk is an age bracket of 50 years or more.

27. The method according to claim 26, wherein said population at greater risk is an age bracket of 60 years or more.

28. The method according to claim 26, wherein said method promotes significant cartilage regeneration, healing or a combination thereof in said subject within six months as compared to microfracture and debridement procedures conducted in subjects in the same age bracket.

29. The method according to claim 25, wherein said population at greater risk is female.

30. The method according to claim 29, wherein said population at greater risk is post-menopausal females.

31. The method according to claim 29, wherein said method promotes significant cartilage regeneration, healing or a combination thereof in said subject within six months as compared to microfracture and debridement procedures conducted in female subjects.

32. The method according to claim 25, wherein said population at greater risk has a body mass index (BMI) of 30 kg/m2 or more.

33. The method according to claim 32, wherein said method promotes significant cartilage regeneration, healing or a combination thereof in said subject within six months of said treatment.

34. The method according to claim 32, wherein said method promotes significant cartilage regeneration, healing or a combination thereof in said subject within twelve months of said treatment.

35. The method according to claim 32, wherein said method promotes significant cartilage regeneration, healing or a combination thereof in said subject within eighteen months of said treatment.

36. The method according to claim 32, wherein microfracture and debridement procedures are contraindicated in said population.

37. The method according to claim 25, wherein said method comprises implanting two or more of said solid substrates.

38. The method according to claim 37, wherein said two or more solid substrates are placed at least at about a 3 mm distance along a cartesian axis from each other.

39. The method according to claim 37, wherein said two or more solid substrates are placed at spaced intervals of at least about 5 mm in distance to span said lesion area larger than 3 cm2.

40. The method according to claim 25, wherein said method promotes cartilage regeneration, healing or a combination thereof even in regions not proximal to said region in which said solid substrates are implanted.

41. The method according to claim 25, wherein said two or more solid substrates are positioned at any relative position with respect to each other within said lesion site.

42. The method according to claim 25, further comprising the step of establishing a specific fluid uptake capacity value of said solid substrate, comprising contacting said solid substrate with a fluid for from 0.1-15 minutes, allowing for spontaneous fluid uptake of said fluid within said solid substrate to arrive at said spontaneous fluid uptake value.

43. The method according to claim 25, further comprising the step of establishing a specific fluid uptake capacity value of said solid substrate comprising contacting said solid substrate with a fluid and applying negative pressure to said solid substrate to promote maximal uptake of said fluid within said solid substrate to arrive at a total fluid uptake value.

44. The method according to claim 25, wherein said subject suffers from a full or partial thickness articular cartilage defect; osteochondral defect; osteoarthritis, a joint defect or a defect resulting from trauma, sports, or repetitive stress.

45. The method according to claim 25, wherein said subject suffers from a bone fracture, bone defect, bone edema, osteoporosis, or a defect resulting from trauma, sports, or repetitive stress.

46. The method according to claim 25, wherein said method serves to resurface an affected joint in a subject.
